(19) United States
(12) Patent Application Publication
(10) Pub. No.: US 2012/0041444 A1
(43) Pub. Date: Feb. 16, 2012

(54) ORTHOPEDIC SURGERIES

(75) Inventor: Thomas A. Einhorn, Boston, MA (US)

(73) Assignee: Warsaw Orthopedic, Inc., Warsaw, IN (US)

(21) Appl. No.: 13/208,608

(22) Filed: Aug. 12, 2011

Related U.S. Application Data
(60) Provisional application No. 61/373,591, filed on Aug. 13, 2010.

(51) Int. Cl. A61B 17/58

(52) U.S. Cl. 606/92

(57) ABSTRACT

The present invention relates to the use of composite materials that can be prepared and delivered to an implant area. The composite materials may be a biocomposite remodeling bone cement (BRBC) material. In one embodiment of the invention the material is introduced in an interface between a first bone and a first joint implant component.

### BACKGROUND

Joint replacement involves replacing painful, arthritic, worn or cancerous joints with artificial implants shaped in such a way as to allow joint movement. In general, there are two strategies for affixing joint replacement implants to the skeleton: cemented and non-cemented. Cemented fixation relies on a stable interface between a prosthesis and a cement, as well as between a cement and bone. Cemented implants may also offer immediate weight-bearing, but potentially poorer final outcome. In contrast, non-cemented devices often have a roughened or porous surface to allow for bone ingrowth and adhesion, which benefits in a long term.

Millions of hip and knee replacements have been performed in America alone over the last four decades. These procedures improve the recipient's range of motion, eliminate pain, and increase activity levels. Although joint replacement surgery has been amazingly successful, some implants will fail and require a second procedure, called revision, to remove the old implants and replace them with new components.

There are several modes of total joint replacement failure. Assuming the device was correctly implanted and there is no post-operative infection, later-time-point failures are often attributed to wear of the articular bearing surfaces (leading to need for replacement of those bearing surfaces) or mechanical loosening (resulting in the need for implant revision surgery). While there may be a variety of reasons for mechanical loosening, it is characterized by dissociation of the interface between bone and a cement, a cement and an implant, or bone and an implant.

### SUMMARY

The present invention encompasses the finding that improvements can be achieved in orthopedic surgeries through the use of composite materials (e.g., biocomposite remodeling bone cement (BRBC) material and other suitable materials). Among other things, the present invention identifies the source of a problem with existing orthopedic surgeries. For example, the present invention encompasses the recognition that cementless bone-to-implant interfaces have the potential to be more reliable than cemented interfaces, as the anticipated ingrowth of bone into the porous implant surface creates a secure bond that is less likely to result in loosening and the need for revision. On the other hand, a tight fit, which helps ensure a positive outcome, is not readily achieved with non-cemented procedures. Thus, the present invention encompasses the recognition that both currently-available approaches to joint fixation are flawed. Among other things, the present invention encompasses the recognition that neither established approach achieves both immediate impact stability and long term resorbability.

The present invention further encompasses recognition that composite materials (e.g., BRBC composite materials) now exist that may have advantageous features of both historical approaches to joint replacement, and may avoid some or all of their disadvantageous features. For example, as appreciated by the present invention, composite materials (e.g., BRBC composite materials) can be delivered and used for initial fixation, and later can go on to permanent fixation by ingrowth. According to the present invention, use of a composite material (e.g., BRBC composite materials) can achieve immediate stability (e.g., after 1-2 days), for example, through fixation of implants to skeleton. In some embodiments, a composite material used in accordance with the present invention has a capacity to stick to bone and metal. Further according to the present invention, use of BRBC composite materials permit resorption and remodeling over time, and even can direct bone ingrowth into implants (e.g., a roughted implant surface). Still further, according to the present invention, using a composite material (e.g., BRBC composite materials) may allow better retention of bone stock. Still further, BRBC composite materials for use in accordance with the present invention, are comprised of compounds selected and arranged so that the composite material achieve at least two physical states—a first state in which the composite material has flowability characteristics appropriate for molding or even injection, and a second state relatively harder than the first state with particular strength; hardness, porosity, resorbability, and/or osteoconductivity characteristics.

The present invention provides new methodologies, tools and/or reagents for orthopedic surgeries, and particularly for surgeries utilizing BRBC composite materials. In some embodiments, the invention provides methodologies, tools, and/or reagents for total knee arthroplasties (TKA). In some embodiments, methods utilized in accordance with the present invention can be performed in a wide variety of joints, which include: knee, hip, ankle, finger, wrist, shoulder and even the elbow. In some embodiments, methods can be used for any joint revision surgeries.

Methods and compositions utilized in accordance with the present invention can be useful in situations in which it is difficult to achieve a close mechanical interface between two surfaces. For example, in a total knee replacement procedure, it can be difficult to achieve a close mechanical interface between a prosthesis and bone (such as a femur or a tibia). As a result, excessive stress can be placed on the prosthesis and/or the bone, which can loosen the bond between the prosthesis and the bone and lead to premature failure. Methods of using compositions in the present invention can strengthen bond between a prosthesis and a bone, for example, by acting as an adhesive or a grout that holds together two or more surfaces and provides a close mechanical interface between surfaces.

Other aspects, features and advantages will be apparent from the description of the embodiments thereof and from the claims.

### BRIEF DESCRIPTION OF DRAWINGS

FIG. 1 illustrates exemplary femur cuts, tibial cuts and patella cuts in total knee anthroplasty (TKA) surgeries.

### DEFINITIONS

The term “adhesive” is used herein to refer to a substance that can cause two or more surfaces to stick together. In some embodiments, an adhesive causes surfaces to stick together through surface contact and without mechanical interference, such as an interference lock. As will be appreciated by these of ordinary skill in the art, cements often do not have adhesive properties while grouts often do. In some embodiments, such a composite material used in the present invention is a cement. In some embodiments, a composite material is a grout. In some embodiments, a composite material has characteristics of both a cement and a grout. The terms “adhesiveness” and “stickiness” as used herein may be interchangeable. In some embodiments, a composite material has a capacity to stick to bone (e.g., tibia, femur, patella) as well as metal (e.g., prosthesis), which contributes to improvements in arthroplasty utilizing the composite material.

The term “bioactive agent” is used herein to refer to compounds or entities that alter, promote, speed, prolong, inhibit, activate, or otherwise affect biological or chemical events in a subject (e.g., a human). For example, bioactive agents may include, but are not limited to osteogenic, osteoinductive, and osteoconductive agents, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug. In certain embodiments, the bioactive agent is a small molecule.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formulary-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21 C.F.R. §§330.5, 331 through 361, and 440 through 460, and drugs for veterinary use listed by the FDA under 21 C.F.R. §§500 through 589, all of which are incorporated herein by reference, are also considered acceptable for use in accordance with the present invention.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “biomolecules” as used herein, refers to classes of molecules (e.g., proteins, amino acids, peptides, polynucleotides, nucleotides, carbohydrates, sugars, lipids, nucleoproteins, glycoproteins, lipoproteins, steroids, natural products, etc.) that are commonly found or produced in cells, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active fragments or subunits. In some embodiments, the biomolecule is a growth factor, chemotactic factor, cytokine, extracellular matrix molecule, or a fragment or derivative thereof, for example, a cell attachment sequence such as a peptide containing the sequence, RGD.

The term “composite” as used herein, is used to refer to a unified combination of two or more distinct materials. A composite may be homogeneous or heterogeneous. For example, a composite may be a combination of particles and a polymer; or a combination of particles, polymers and antibiotics. In certain embodiments, a composite has a particular orientation.

The term “demineralized” is used herein to refer to particles (e.g., bone particles) that have been subjected to a process that causes a decrease in the original mineral content. As utilized herein, the phrase “superficially demineralized” as applied to bone particles refers to bone particles possessing at least about 90% by weight of their original inorganic mineral content. The phrase “partially demineralized” as applied to the bone particles refers to bone particles possessing from about 8% to about 90% by weight of their original inorganic mineral content, and the phrase “fully demineralized” as applied to the bone particles refers to bone particles possessing less than about 8% by weight, for example, less than about 1% by weight, of their original inorganic mineral content. The unmodified term “demineralized” as applied to the bone particles is intended to cover any one or combination of the foregoing types of demineralized bone particles.

The term “deorganified” as herein applied to matrices, particles, etc., refers to bone or cartilage matrices, particles, etc., that were subjected to a process that removes at least part of their original organic content. In some embodiments, at least 1%, 10%, 20%, 30%, 40%, 50%, 60%, 70%, 80%, 90%, or 99% of the organic content of the starting material is removed. Deorganified bone from which substantially all the organic components have been removed is termed “anorganic.”

The term “flowable polymer material” as used herein, refers to a flowable composition including one or more of monomers, pre-polymers, oligomers, low molecular weight polymers, uncross-linked polymers, partially cross-linked polymers, partially polymerized polymers, polymers, or combinations thereof that have been rendered formable. One skilled in the art will recognize that a flowable polymer material need not be a polymer but may be polymerizable. In some embodiments, flowable polymer materials include polymers that have been heated past their glass transition or melting point. Alternatively or in addition, a flowable polymer material may include partially polymerized polymer, telechelic polymer, or prepolymer. A pre-polymer is a low molecular weight oligomer typically produced through step growth polymerization. The pre-polymer is formed with an excess of one of the components to produce molecules that are all terminated with the same group. For example, a diol and an excess of a diisocyanate may be polymerized to produce isocyanate terminated prepolymer that may be combined with a diol to form a polyurethane. Alternatively or in addition, a flowable polymer material may be a polymer material/solvent mixture that sets when the solvent is removed.

The term “mineralized” as used herein, refers to bone that has been subjected to a process that caused a decrease in their original organic content (e.g., de-fatting, de-greasing). Such a process can result in an increase in the relative inorganic mineral content of the bone. Mineralization may also refer to the mineralization of a matrix such as extracellular matrix or demineralized bone matrix. The mineralization process may take place either in vivo or in vitro.

The term “non-demineralized” as herein applied to bone or bone particles, refers to bone or bone-derived material (e.g., particles) that have not been subjected to a demineralization process (i.e., a procedure that totally or partially removes the original inorganic content of bone).

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The term “osteo conductive” as used herein, refers to the ability of a substance or material to provide surfaces which are receptive to the growth of new bone.

The term “osteogenic” as used herein, refers to the ability of a substance or material that can induce bone formation.

The term “osteoinductive” as used herein, refers to the quality of being able to recruit cells (e.g., osteoblasts) from the host that have the potential to stimulate new bone formation. In general, osteoinductive materials are capable of inducing heterotopic ossification, that is, bone formation in extraskeletal soft tissues (e.g., muscle).

The term “osteoimplant” is used herein in its broadest sense and is not intended to be limited to any particular shapes, sizes, configurations, compositions, or applications. Osteoimplant refers to any device or material for implantation that aids or augments bone formation or healing. Osteoimplants are often applied at a bone defect site, e.g., one resulting from injury, defect brought about during the course of surgery, infection, malignancy, inflammation, or developmental malformation. Osteoimplants can be used in a variety of orthopedic, neurosurgical, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc.

The term “polyurethane,” as used herein, is intended to include all polymers incorporating more than one urethane group (—NH—CO—O—) in the polymer backbone. Polyurethanes are commonly formed by the reaction of a polyisocyanate (such as a diisocyanate) with a polyol (such as a diol):

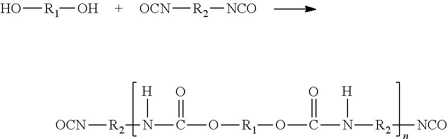

The term “porogen” as used herein, refers to a chemical compound that may be part of a composite material and upon implantation/injection or prior to implantation/injection diffuses, dissolves, and/or degrades to leave a pore in the osteoimplant composite. A porogen may be introduced into a composite material during manufacture, during preparation of composite materials (e.g., in the operating room), or after implantation, delivery and/or injection of composite materials. A porogen essentially reserves space in a composite material while the composite material is being molded but once the composite is implanted the porogen diffuses, dissolves, or degrades, thereby inducing porosity into composite materials. In this way porogens provide latent pores. In certain embodiments, a porogen may be leached out of the composite before implantation, delivery and/or injection. This resulting porosity of the implant generated during manufacture or after implantation, delivery and/or injection (i.e., “latent porosity”) is thought to allow infiltration by cells, bone formation, bone remodeling, osteoinduction, osteoconduction, and/or faster degradation of the osteoimplant. A porogen may be a gas (e.g., carbon dioxide, nitrogen, or other inert gas), liquid (e.g., water, biological fluid), or solid. Porogens are typically water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade quickly under physiological conditions. Exemplary polymers include polyethylene glycol, poly(vinylpyrollidone), pullulan, poly(glycolide), poly(lactide), poly(lactide-co-glycolide), other polyesters, and starches. In certain embodiments, bone particles utilized in composite materials or compositions act as porogens. For example, osteoclasts resorb allograft and make pores in composite materials. In some embodiments, porogens may refer to a blowing agent (i.e., an agent that participates in a chemical reaction to generate a gas). Water may act as such a blowing agent or porogen.

The term “porosity” as used herein, refers to the average amount of non-solid space contained in a composite material (e.g., BRBC composite materials used in the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composite materials. In some embodiments, porosity (c), defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation, delivery and/or injection. It will be appreciated by these of ordinary skill in the art that porosity of a composite material or composition may change over time, in some embodiments, after implantation, delivery and/or injection (e.g., after leaching of a porogen, when osteoclasts resorbing allograft bone, etc.). In some embodiments, a composite material (e.g., BRBC composite materials) to be utilized in accordance with the present invention having a porosity of less than 2 vol %, less than 5 vol %, less than 10 vol %, less than 15 vol %, less than 20 vol %, less than 30 vol %, less than 40 vol %, less than 50 vol %, less than 60 vol %, less than 70 vol %, less than 90 vol % or at least about 90 vol %, before being hardened. For the purpose of the present disclosure, the beginning of mixing of components of such composite materials utilized in the present invention may be considered to be “time zero” (T₀). In some embodiments, composite materials (e.g., BRBC composite materials) have a porosity of as low as 1 vol % or 2 vol % at time zero. In some embodiments, composite materials cure in situ and have a porosity of less than 2 vol %, less than 5 vol %, less than 10 vol %, less than 15 vol %, less than 20 vol %, less than 30 vol %, less than 40 vol %, less than 50 vol %, less than 60 vol %, less than 70 vol %, less than 90 vol % or at least about 90 vol %, after being hardened fully. At the end of hardening of such composite materials, when viscosity of composite materials reaches a certain value and levels off (e.g., when components of the composite complete polymerization) may be considered to be “time end” (Tₕ).

The term “remodeling” as used herein, describes the process by which native bone, processed bone allograft, whole bone sections employed as grafts, and/or other bony tissues are replaced with new cell-containing host bone tissue by the action of osteoclasts and osteoblasts. Remodeling also describes the process by which non-bony native tissue and tissue grafts are removed and replaced with new, cell-containing tissue in vivo. Remodeling also describes how inorganic materials (e.g., calcium-phosphate materials, such as β-tricalcium phosphate) is replaced with living bone.

The term “setting time” as used herein, is approximated by the tack-free time (TFT), which is defined as the time at which a material could be touched with a spatula with no adhesion of the spatula to the foam of the material. At the TFT, wound could be closed without altering properties of a material. The terms “set” and “harden” as used herein may be interchangeable.

The term “shaped” as used herein, is intended to characterize a material (e.g., composite material) or an osteoimplant refers to a material or osteoimplant of a determined or regular form or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, bones, portions of bones, wedges, cylinders, threaded cylinders, and the like, as well as more complex geometric configurations.

The term “transformation” as used herein, describes a process by which a material is removed from an implant site and replaced by host tissue after implantation. Transformation may be accomplished by a combination of processes, including but not limited to remodeling, degradation, resorption, and tissue growth and/or formation. Removal of the material may be cell-mediated or accomplished through chemical processes, such as dissolution and hydrolysis.

The term “wet compressive strength” as used herein, refers to the compressive strength of an osteoimplant after being immersed in physiological saline (e.g., phosphate-buffered saline (PBS), water containing 0.9 g NaCl/100 ml water, etc.) for a minimum of 12 hours (e.g., 24 hours). Compressive strength and modulus are well-known measurements of mechanical properties and is measured using the procedure described herein

The term “working time” as used herein, is defined in the ISO9917 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows. According to the present invention, the working time is measured by loading the syringe with the reactive composite material and injecting <0.25 ml every 30 s. The working time is noted as the time at which the material was more difficult to inject, indicating a significant change in viscosity.

The term “load-bearing” as used herein, refers to the ability of a material to bear weight and force resting upon it, conducting a vertical load from the upper structure to the foundation. Measurements of stiffness may be used as a promising tool for indicating load-bearing capacity. In this context, compressive strength may be referred to characterize the load-bearing capacity of a material (e.g., hardened composite materials). For example, a material having wet compressive strength of 0.1 MPa or more than 0.1 MPa is considered load-bearing, while having wet compressive strength of less than 0.1 MPa is non-load-bearing. In some embodiments, a material having wet compressive strength of more than 0.5 MPa, 1 MPa, or 3 MPa is load-bearing. Correspondingly, a material having wet compressive strength of less than 0.5 MPa, 1 MPa, or 3 MPa is non-load-bearing in some embodiments. In some embodiments, composite materials (e.g., BRBC composite materials and other suitable materials) utilized in accordance with the present invention are load-bearing. In certain embodiments, they are non-load-bearing.

### DETAILED DESCRIPTION OF CERTAIN EMBODIMENTS

As used herein and in the appended claims, the singular forms “a,” “an” and “the” include plural references unless the content clearly dictates otherwise. All publications, patent applications, patents, and other references mentioned herein are incorporated by reference in their entirety.

Methodologies, tools and/or reagents for orthopedic surgeries disclosed herein can be used in a variety of reconstructive orthopedic surgical procedures, such as joint arthroplasties. Specific examples include total knee arthroplasty (TKA) or revision, total hip replacement or revision, total shoulder arthroplasty or revision, and other procedures involving large joints. In some embodiments, methods utilized in accordance with the present invention can be performed in a wide variety of joints, which include: knee, hip, ankle, finger, wrist, shoulder and even the elbow. In some embodiments, methods can be used for any joint revision surgeries.

Composite materials to be utilized in accordance with the present invention such as Biocomposite Remodeling Bone Cement (BRBC) materials and other suitable materials utilized in accordance with the present invention are capable of providing initial fixation and stabilization of prosthetic implants or components, and conducting bone remodeling and biological in-growth for implants. In some embodiments, a composite material (e.g., a BRBC composite material) serve as a good adhesive between a prosthesis and bone, thereby providing a close mechanical interface between a prosthesis and bone. By allowing and encouraging direct boney in-growth and remodeling, use of composite materials can reduce potential loosening and failure of a prosthesis, and improve patient outcome.

### Orthopedic Surgical Procedures

The replacement of diseased or injured joints (arthroplasty) by artificial materials has become one of the more practiced procedures in orthopaedic surgery. The primary cause leading to total joint replacements is degenerative joint disease which leads to erosion of the articular cartilage layer and subsequent pain. A non-cemented procedure to replace diseased or injured joints may involve implanting an allograft bone or a coated device including those disclosed in U.S. Pat. No. 5,061,286, the content of which is corporate by reference, and those discussed in Hofmann et al. International Orthopaedics, (SICOT) 1992, 16: 340-358 and Bloebaum et al. the Journal of Arthroplasty Vol. 7, No. 4 1992, 483-493. However, roughened or porous surface of non-cemented prosthesis allows for bone ingrowth and adhesion. This ability for bone to grow into the surface of the prosthesis is highly dependant upon the closeness of fit between the implant and bone surface. Unfortunately, a tight fit with no gaps which helps to ensure a positive outcome is not readily achieved in non-cemented procedures. The bonding between a prosthesis and bone, in a non-cemented arthroplasty, is dependent upon bony ingrowth, while a bone cement (e.g., PMMA) is applied at the time of surgery in a cemented arthroplasty, forming a solid bond between a prosthesis and bone. Potential advantages of cementing include firm fixation and a reduced long-term revision rate from loosening of the prosthesis. Major side effects of cements, however, include cardiac arrhythmias and cardio-respiratory collapse, which occasionally occur on applications. These potentially fatal complications are caused either by embolism from marrow contents forced into the circulation or by a direct toxic effect of the cement. Another major disadvantage of a cemented prosthesis is that revision arthroplasty will be more difficult. See, Vochteloo et al., BMC Muschloskeletal Disorders 2009, 10: 56.

The present invention encompasses the recognition that available non-cemented approaches fail to achieve immediate stability; moreover, the present invention further encompasses the recognition that current cementing approaches fail to achieve long term stability due, among other things, to the lack of remodeling.

In recognition to problems in arthroplasty, in particular, total arthroplasty, inventive methods using certain composite materials (e.g., BRBC composite materials) are disclosed herein. Provided technologies can achieve close contact, initial stability, resorbability, bone ingrowth, and long-term resorption.

In some embodiments, use of a composite material (e.g., BRBC composite materials) in accordance with the present invention reduces rates of revision arthroplasty as compared with that observed with standard cements (e.g., PMMA). A revision joint replacement, also called a revision arthroplasty, is required to replace a worn out joint replacement due to worn-out implants, infection of a replaced joint, and instability or malpositioning of an implanted joint. The failure of implants may be compounded by bone lost during removal of the failed implants. Substantial bone lost and bone defects are among the most challenging problems faced by surgeons performing revision surgery. It is important, particularly in a young patient, to minimize bone loss and try to restore bone stock. Inventive methods utilized in accordance with the present invention using certain composite materials (e.g., BRBC composite materials) also provide a solution for restoration of bone stock by encouraging bone ingrowth.

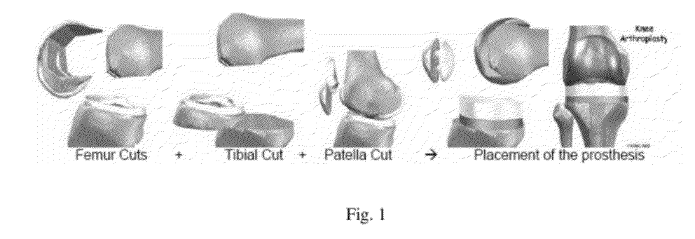

A total knee arthroplasty (TKA) surgery, for example, is extremely technique dependant. A surgeon is required to make multiple cuts to the femur, tibia and patella to match the bone surfaces to the implant shape, shown in FIG. 1. It may be difficult to sculpt a perfect fit and achieve a close mechanical interface between the knee prostheses and bone. For this reason, a majority (nearly 90%) of knee implants are cemented. Cement is used as a grout, to fill any gaps or voids between implants (e.g., allograft or metal prosthesis) and patient bone.

Before 1940s, knee arthroplasty was limited to partial replacements of the joint surfaces and hinge designs that relied upon ligament stability and simple bone-metal contact to keep the prosthetic device in the planned position. In the early 1960's, polymethylmethacrylate (PMMA) bone cement was introduced into the emerging field of total joint replacements. PMMA is a derivative of acrylic acid that is formed by the combination of monomer liquid mixed with polymer powder that leads to an exothermic reaction with change into a solid state. The solid power consists of polymethylmethacrylate polymer and methylmethacrylate styrene copolymer. The liquid monomer (methylmethacrylate) leads to polymerization and bonds the spherical copolymer molecules in a polymethylmethacrylate matrix. Today, PMMA is widely used in cemented TKA, providing adjunctive fixation of the femoral stem to the adjacent bone. However, significant problems do exist.

For decades, surgeons have been using PMMA as a bone cement, which cures in the body at high temperatures, capable of causing necrosis to surrounding tissue. In addition, previous treatment options presented environmental hazards to medical personal due to the toxic fumes released during mixing and preparation. Furthermore, one constituent of the liquid monomer is N,N-Dimethyl-Para-Toluidine (DMPT), a toxic material. Other monomer ingredients also exhibit adverse effects on humans. Thus, the introduction of the mixed, but yet unset, PMMA, into interfaces between implants and bones, presents the potential of introducing a significant amount of toxic material into the blood stream, especially when locating the stems of the prosthesis into the medullary canal. Various reactions can occur to PMMA cement, such as hypotension and even circulatory system collapse.

Furthermore, as described herein the present invention encompasses the recognition that PMMA alone is not osteogenic, osteoinductive or osteoconductive, so that a truly solid bond between a prosthesis and bone does not form; a revision arthroplasty under such circumstances can be much more difficult. The present invention provides the insight that certain composite materials (e.g., BRBC composite materials) are osteogenic, osteoinductive or osteoconductive, and furthermore can achieve both immediate stability and long term resorption. Among other things, provided strategies can achieve close contact between implant and site, and reduced rates of revision surgery as compared with standard cements such as PMMA.

The present invention provides methods that utilize composite materials (e.g., BRBC composite materials) as disclosed herein, and avoids issues with cement materials such as PMMA. Cement materials utilized in accordance with the present invention can be filled into any gaps between bone and prosthesis and encourage bone ingrowth into the prosthesis surface—combining the forgiving elements of a cemented joint replacement with the bone ingrowth benefits of a cementless construct. Not only can certain composite materials (e.g., BRBC composite materials) utilized in accordance with the present invention provide a custom fit of a prosthesis into a bone cavity, but also can act as a buffering layer, distributing the applied stresses from the stiff prosthetic stem to the more compliant bone. Furthermore, direct boney ingrowth with a prosthesis would help to mitigate potential loosening, improving patient outcome.

In some embodiments, methods utilized in accordance with the present invention can be performed in a wide variety of joints, which include: knee, hip, ankle, finger, wrist, shoulder and even the elbow. At early stage of joint replacements, loosening is often a result of misshapen, cuts, or poor bone/site preparation leading to a mismatch at the interface of implant and patient bone. At later stage, loosening is often a result of poor patient bone quality. In some embodiments, methods utilized in accordance with the present invention can also be used in any joint revision surgeries.

In general, suitable materials, for example, BRBC composite materials utilized in accordance with the present invention may replace PMMA in arthroplasty procedures. In some embodiments, a composite material (e.g., BRBC composite materials) is used to supplement a cementless implant system in TKA. It can fill in gaps between low spots of bone bed and an implant. The implant can be placed with as much direct bone contact/support as possible. Although most existing TKA implants are non-porous, a matter or rough surface of implants in some embodiments can be used with a composite material (e.g., BRBC composite materials). In TKA, a composite material may be used on the tray of a tibial side. In certain embodiments, composite materials (e.g., BRBC composite materials) are less viscous such that initial interdigitation with the trabecular structure on a tibial side is obtained. In certain embodiments, composite materials (e.g., BRBC composite materials) are used to address difficulty with press fit. On a femoral side, they can be used as more of a grout being more viscous. In certain embodiments, composite materials (e.g., BRBC composite materials) are be used on patella, where initial stability and confidence that materials will remodel is needed. Viscosity of composite materials used on patella may be higher than their initial states.

As will be appreciated by one of skill in the art, in some embodiments, a composite material (e.g., BRBC composite materials) including a particular component, a polymer, and optionally, one or more additives can be prepared in an operating room, which allows a surgeon to tailor composition according to a selected application. A composite material can be porous as-prepared and/or porosity of a composite can change (e.g., increase) over time to support in-growth of bone. Over the course of a selected time period (e.g., weeks, months or years), a composite material (e.g., at a hardened state) can remodel into host bone. For example, cancellous bone incorporated into polymer precursors can be exposed at a site of a implant to provide access to osteoprogenitor cells. Furthermore, viscosity or consistency of a composite material may change from when it is initially prepared (e.g., in a flowable state). For example, a composite material can initially have a flowable, liquid-like consistency, which allows it to be easily injected and applied to certain surfaces such as on a prosthesis or a bone. Flowable consistency also allows a composite material (e.g., a flowable cement) to penetrate trabeculae of bone. Over the course that a composite material is handled (e.g., 2-5 minutes), it can become more viscous (e.g., dough-like), which allows it to act as a grout (e.g., to fill gaps, holes, and defects from sub-millimeter to 3 mm) and to be moldable by hand. Increase in viscosity can also provide a composite material adhesive strength to fix and to stabilize a prosthesis until a hardened composite is resorbed and/or remodeling or in-growth occurs (e.g., over approximately 0.2, 0.3, 0.5, 1 or 2 years). In some embodiments, such as a composite material can be mixed with antibiotics, cells, growth factors, etc, so that a hardened cement including bioactive agents can prevent infection, and/or improve bone healing. After a cement is applied, it can harden substantially to serve as a load-bearing material. A hardened composite is not moldable by hand and not noticeably affected by heat or irrigation. In other embodiments, a hardened composite is a non-load-bearing material.

In some embodiments, a composite material (e.g., BRBC composite materials) utilized in accordance with the present invention can be load-bearing materials. In some embodiments, certain composite materials (e.g., BRBC composite materials) for use in accordance with the present invention are also used as non-load-bearing bone void fillers either alone or in combination with one or more other conventional devices.

Various composite materials (e.g., BRBC composite materials and other suitable materials) can be used in accordance with the present invention. In some embodiments, composite materials include particles combined with polymers as disclosed in U.S. Pat. No. 7,291,345, filed Dec. 12, 2003; U.S. patent application Ser. No. 11/934,980, filed Nov. 5, 2007, and published under publication number 20080063684; U.S. patent application Ser. No. 11/047,992, filed Jan. 31, 2005, and published under publication number 200600015184; and U.S. patent application Ser. No. 11/625,119, filed Jan. 19, 2007, and published under publication number 2007/0191963; each of which is incorporated herein by reference. In some embodiments, composite materials are materials described in U.S. patent application Ser. No. 11/625,086, filed Jan. 19, 2007, and published under publication number 20080069852; each of which is incorporated herein by reference.

In some embodiments, composite materials are materials described in U.S. Pat. No. 6,294,187, filed Feb. 23, 1999; U.S. Pat. No. 6,440,444, filed Jul. 24, 2001; U.S. Pat. No. 6,696,073, filed Aug. 27, 2002; U.S. patent application Ser. No. 10/736,799, filed Dec. 16, 2003, and published under publication number 20080188945; U.S. patent application Ser. No. 11/758,751, filed Jun. 6, 2007, and published under publication number 20070233272; each of which is incorporated herein by reference.

In some embodiments, composite materials are heat sensitive. In some embodiments, composites are a material involving heating, such as Plexur M™ from Osteotech—one that would soften when heated, then harden at body temperature.

In some embodiments, composite materials are not heat sensitive, for example, polyurethane-based composite materials may have a low reaction exotherm. In some embodiments, composite materials are polyurethane-based materials disclosed in U.S. patent application Ser. No. 10/771,736 and U.S. patent application Ser. No. 12/608,850; each of which is incorporated by references. In some embodiments, composite materials are materials disclosed in Guelcher et al, Synthesis and characterization of an injectable allograft bone/polymer composite bone void filler with tunable mechanical properties, Tissue Engineering, Part A, 2010 in press; Guelcher et al, Synthesis, characterization, and remodeling of weight-bearing allograft bone/polyurethane composites in the rabbit, Acta Biomaterialia, 2010 in press; Guelcher et al., Tissue Eng 2006; 12(5):1247-1259; Hafeman et al., Pharm Res 2008; 25(10):2387-99; Guelcher et al., Tissue Engineering 2007; 13(9):2321-2333; Guelcher, Tissue Engineering: Part B, 14 (1) 2008, pp 3-17; each of which is incorporated by references.

Composite materials utilized the present application may contain porogens or no porogens. In some embodiments, composite materials are or include KRYPTONITE™, a non-toxic bone cement, with bone-like mechanical properties composed of naturally occurring fatty acids and calcium carbonate. In some embodiments, composite materials are or include Stryker bone cements (e.g., Simplex P). The ingredients of Simplex P can be methylmethacrylate-styrene copolymer for strength, PMMA for handling, barium sulfate for radiopaqueness and benzoyl peroxide. In some embodiments, composite materials are mixed with an additive such as antibiotics (e.g., tobramycin) and bone marrow aspirate concentrate (BMAC). In some embodiments, other property-enhancing ingredients may be included and, optionally, evenly mixed into a composite material for delivery.

### Composite Materials and Biocomposite Remodeling Bone Cement (BRBC) Materials

One aspect of the present invention is the recognition that certain composite materials (e.g., BRBC composite materials) are particularly useful in the context of orthopedic surgical procedures. Composite materials (e.g., BRBC composite materials) can be used to fill any gaps between bone and a prosthesis and encourage bone ingrowth into prosthesis surface. According to the present invention, methods utilizing composite materials (e.g., BRBC composite materials) provide a hybrid form of cemented and non-cemented arthoplastry.

Materials/compositions used in accordance with the present invention adapt at least a first state in which they are injectable, moldable and/or flowable enough to be delivered. In some embodiments, materials/compositions are provided in their flowable state to an implant site and then set into a second, hardened state, thereby fixing implant components to bone.

Particulate Component

Particles used in accordance with the present invention may include a bone-derived material, an inorganic material, a bone substitute material, a composite material, or any combinations thereof.

Bone Particles. Any kind of bone and/or bone-derived particles may be used in the present invention. In some embodiments, bone particles employed in the preparation of bone particle-containing composite materials are obtained from cortical, cancellous, and/or corticocancellous bone. Bone particles may be obtained from any vertebrate. Bone may be of autogenous, allogenic, and/or xenogeneic origin. In certain embodiments, bone particles are autogenous, that is, bone particles are from the subject being treated. In other embodiments, bone particles are allogenic (e.g., from donors). In certain embodiments, source of bone may be matched to the eventual recipient of composite materials (i.e., the donor and recipient are of the same species). For example, human bone particle is typically used in a human subject. In certain embodiments, bone particles are obtained from cortical bone of allogenic origin. In certain embodiments, bone particles are obtained from bone of xenogeneic origin. Porcine and bovine bone are types of xenogeneic bone tissue that can be used individually or in combination as sources for bone particles and may offer advantageous properties. Xenogenic bone tissue may be combined with allogenic or autogenous bone.

Bone particles can be formed by any process known to break down bone into small pieces. Exemplary processes for forming such particles include milling whole bone to produce fibers, chipping whole bone, cutting whole bone, grinding whole bone, fracturing whole bone in liquid nitrogen, or otherwise disintegrating the bone. Bone particles can optionally be sieved to produce particles of a specific size range. Bone particles may be of any shape or size. Exemplary shapes include spheroidal, plates, shards, fibers, cuboidal, sheets, rods, oval, strings, elongated particles, wedges, discs, rectangular, polyhedral, etc.

In some embodiments, bone particles have a medium or mean diameter about 1200 microns, 1100 microns, 1000 microns, 900 microns, 800 microns, 700 microns, 600 microns, 500 microns, 400 microns, 300 microns, 200 microns, 100 microns, etc. In some embodiments, diameters of bone particles are within a range between any of such sizes. For example, medium or mean diameters of bone particles have a range from approximately 100 microns to approximately 1000 microns.

As for irregularly shaped bone particles, recited dimension ranges may represent the length of the greatest or smallest dimension of the particle. As examples, bone particles can be pin shaped, with tapered ends having an average diameter of from about 100 microns to about 500 microns. As will be appreciated by one of skill in the art, for injectable composite materials, the maximum particle size will depend in part on the size of the cannula or needle through which the material will be delivered.

In some embodiments, particle size distribution of bone particles utilized in accordance with the present inventions with respect to a mean value or a median value may be plus or minus, e.g., about 10% or less of the mean value, about 20% or less of the mean value, about 30% or less of the mean value, about 40% or less of the mean value, about 50% or less of the mean value, about 60% or less of the mean value, about 70% or less of the mean value, about 80% or less of the mean value, or about 90% or less of the mean value.

In some embodiments, bone particles have a median or mean length of about 1200 microns, 1100 microns, 1000 microns, 900 microns, 800 microns, 700 microns, 600 microns, 500 microns, 400 microns, 300 microns, 200 microns, 100 microns, etc. In some embodiments, about 70, about 80 or about 90 percent of bone particles possess a median or mean length within a range of any of such sizes.

For bone particles that are fibers or other elongated particles, in some embodiments, at least about 90 percent of the particles possess a median or mean length in their greatest dimension in a range from approximately 100 microns to approximately 1000 microns. Particles may possess a median or mean length to median or mean thickness ratio from at least about 5:1 up to about 500:1, for example, from at least about 50:1 up to about 500:1, or from about 50:1 up to about 100:1; and a median or mean length to median or mean width ratio of from about 10:1 to about 200:1 and, for example, from about 50:1 to about 100:1. In certain embodiments, bone particles are short fibers having a cross-section of about 300 microns to about 100 microns and a length of about 0.1 mm to about 1 mm.

Processing of bone to provide particles may be adjusted to optimize for the desired size and/or distribution of bone particles. Properties of resulting composite materials (e.g., mechanical properties) may also be engineered by adjusting weight percent, shapes, sizes, distribution, etc. of bone particles or other particles. For example, a composite material may be made more viscous and load bearing by including a higher percentage of particles.

U.S. Pat. Nos. 5,899,939; 5,507,813; 6,123,731; 6,294,041; 6,294,187; 6,332,779; 6,440,444; and 6,478,825; the contents of all of which are incorporated herein by reference, describe methods for preparing composite materials including allogenic bone for use in orthopedic applications.

Bone particles utilized in accordance with the present inventions may be demineralized, non-demineralized, mineralized, or anorganic. In some embodiments, bone particles are used “as is” in preparing composite materials. In some embodiments, bone particles are defatted and disinfected. An exemplary defatting/disinfectant solution is an aqueous solution of ethanol. Other organic solvent may also be used in the defatting and disinfecting bone particles. For example, methanol, isopropanol, butanol, DMF, DMSO, diethyl ether, hexanes, glyme, tetrahydrofuran, chloroform, methylene chloride, and carbon tetrachloride may be used. In certain embodiments, a non-halogenated solvent is used. A defatting/disinfecant solution may also include a detergent (e.g., an aqueous solution of a detergent). Ordinarily, at least about 10 to about 40 percent by weight of water (i.e., about 60 to about 90 weight percent of defatting agent such as alcohol) should be present in the defatting/disinfecting solution to produce optimal lipid removal and disinfection within the shortest period of time. An exemplary concentration range of a defatting solution is from about 60 to about 85 weight percent alcohol, for example, about 70 weight percent alcohol.

In some embodiments, bone particles are demineralized. Bone particles can be optionally demineralized in accordance with known and/or conventional procedures in order to reduce their inorganic mineral content. Demineralization methods remove the inorganic mineral component of bone by employing acid solutions. Such methods are well known in the art, see for example, Reddi, et al., Proc. Nat. Acad. Sci., 1972, 69:1601-1605, the contents of which are incorporated herein by reference. The strength of the acid solution, the shape and dimensions of the bone particles and the duration of the demineralization treatment will determine the extent of demineralization. Reference in this regard is made to Lewandrowski, et al., J. Biomed. Mater. Res., 1996, 31:365-372 and U.S. Pat. No. 5,290,558, the contents of both of which are incorporated herein by reference.

In an exemplary defatting/disinfecting/demineralization procedure, bone particles are subjected to a defatting/disinfecting step, followed by an acid demineralization step. An exemplary defatting/disinfectant solution is an aqueous solution of ethanol. In some embodiments, at least about 10 to about 40 percent by weight of water (i.e., about 60 to about 90 weight percent of defatting agent such as alcohol) can be present in a defatting/disinfecting solution to produce optimal lipid removal and disinfection within a reasonable period of time. An exemplary concentration range of a defatting solution is from about 60 to about 85 weight percent alcohol, for example, about 70 weight percent alcohol. Ethanol is typically the alcohol used in this step; however, other alcohols such as methanol, propanol, isopropanol, denatured ethanol, etc. may also be used. Following defatting, bone particles can be immersed in acid over time to effect their demineralization. The acid also disinfects the bone by killing viruses, vegetative microorganisms, and spores. Acids which can be employed in this step include inorganic acids such as hydrochloric acid and organic acids such as peracetic acid. After acid treatment, demineralized bone particles can be rinsed with sterile water to remove residual amounts of acid and thereby raise the pH. Bone particles may be dried, for example, by lyophilization, before being incorporated into a composite material. Bone particles may be stored under aseptic conditions, for example, in a lyophilized state, until they are used or sterilized using known methods (e.g., gamma irradiation) shortly before combining them with polyurethanes used in composite materials.

As utilized herein, the phrase “superficially demineralized” as applied to the bone particles refers to bone particles possessing at least about 90% by weight of their original inorganic mineral content. The phrase “partially demineralized” as applied to the bone particles refers to bone particles possessing from about 8% to about 90% weight of their original inorganic mineral content, and the phrase “fully demineralized” as applied to the bone particles refers to bone particles possessing less than about 8%, preferably less than about 1%, by weight of their original inorganic mineral content. The unmodified term “demineralized” as applied to the bone particles is intended to cover any one or combination of the foregoing types of demineralized bone particles, that is, superficially demineralized, partially demineralized, or fully demineralized bone particles.

In alternative embodiments, surfaces of bone particles may be lightly demineralized according to the procedures in our commonly owned U.S. patent application, U.S. Ser. No. 10/285,715, filed Nov. 1, 2002, published as U.S. Patent Publication No. 2003/0144743, on Jul. 31, 2003, the contents of which are incorporated herein by reference. Even minimal demineralization, for example, of less than 5% removal of the inorganic phase, increases the hydroxylation of bone fibers and the surface concentration of amine groups. Demineralization may be so minimal, for example, less than 1%, that the removal of the calcium phosphate phase is almost undetectable. Rather, the enhanced surface concentration of reactive groups defines the extent of demineralization. This may be measured, for example, by titrating the reactive groups. Surface composition can also be measured by x-ray photoelectron spectroscopy (XPS), an experimental technique that measures the atomic composition of the top 1-10 nm of the surface. In some embodiments, in a polymerization reaction that utilizes the exposed allograft surfaces to initiate a reaction, the amount of unreacted monomer remaining may be used to estimate reactivity of the surfaces. Surface reactivity may be assessed by a surrogate mechanical test, such as a peel test of a treated coupon of bone adhering to a polymer.

In certain embodiments, bone particles are subjected to a process that partially or totally removes their initial organic content to yield mineralized and anorganic bone particles, respectively. Different mineralization methods have been developed and are known in the are (Hurley, et al., Milit. Med. 1957, 101-104; Kershaw, Pharm. J. 6:537, 1963; and U.S. Pat. No. 4,882,149; each of which is incorporated herein by reference). For example, a mineralization procedure can include a de-greasing step followed by a basic treatment (with ammonia or another amine) to degrade residual proteins and a water washing (U.S. Pat. Nos. 5,417,975 and 5,573,771; both of which are incorporated herein by reference). Another example of a mineralization procedure includes a defatting step where bone particles are sonicated in 70% ethanol for 1-3 hours.

In some embodiments, bone particles can be modified in one or more ways, e.g., their protein content can be augmented or modified as described, for example, in U.S. Pat. Nos. 4,743,259 and 4,902,296, the contents of both of which are incorporated herein by reference.

Mixtures or combinations of one or more of the foregoing types of bone particles can be employed. For example, one or more of the foregoing types of demineralized bone particles can be employed in combination with non-demineralized bone particles, i.e., bone particles that have not been subjected to a demineralization process, or inorganic materials. The amount of each individual type of bone particle employed can vary widely depending on the mechanical and biological properties desired. Thus, in some embodiments, mixtures of bone particles of various shapes, sizes, and/or degrees of demineralization may be assembled based on the desired mechanical, thermal, chemical, and biological properties of a composite material. A desired balance between the various properties of composite materials (e.g., a balance between mechanical and biological properties) may be achieved by using different combinations of particles. Suitable amounts of various particle types can be readily determined by those skilled in the art on a case-by-case basis by routine experimentation.

The differential in strength, osteogenicity, and other properties between partially and fully demineralized bone particles on the one hand, and non-demineralized, superficially demineralized bone particles, inorganic ceramics, and other bone substitutes on the other hand can be exploited. For example, in order to increase the compressive strength of an osteoimplant, the ratio of nondemineralized and/or superficially demineralized bone particles to partially or fully demineralized bone particles may favor the former, and vice versa. Bone particles in composite materials also play a biological role. Non-demineralized bone particles bring about new bone in-growth by osteoconduction. Demineralized bone particles likewise play a biological role in bringing about new bone in-growth by osteoinduction. Both types of bone particles are gradually remodeled and replaced by new host bone as degradation of the composite progresses over time. Thus, the use of various types of bone particles can be used to control the overall mechanical and biological properties, (e.g., strength, osteoconductivity, and/or osteoinductivity, etc.) of osteoimplants.

Surface Modification. Bone particles utilized in accordance with the present invention may be optionally treated to enhance their interaction with polymer components (e.g., prepolymers of polyurethanes) and/or to confer some properties to particle surface. While some bone particles will interact readily with monomers and be covalently linked to polyurethane matrices, it may be desirable to modify surface of bone particles to facilitate their incorporation into polymers that do not bond well to bone, such as poly(lactides). Surface modification may provide a chemical substance that is strongly bonded to the surface of bone, e.g., covalently bonded to the surface. Bone particles may, alternatively or additionally, be coated with a material to facilitate interaction with polymers of composite materials.

In some embodiments, silane coupling agents are employed to link a monomer or initiator molecule to the surface of bone particles. Silane has at least two sections, a set of leaving groups and at least an active group. An active group may be connected to the silicon atom in the silane by an elongated tether group. An exemplary silane coupling agent is 3-trimethoxysilylpropylmethacrylate, available from Union Carbide. Three methoxy groups are leaving groups, and the methacrylate active group is connected to the silicon atom by a propyl tether group. In some embodiments, a leaving group is an alkoxy group such as methoxy or ethoxy. Depending on the solvent used to link the coupling agent to bone particles, hydrogen or alkyl groups such as methyl or ethyl may serve as leaving groups. The length of tethers determines the intimacy of connection between polymers and bone particles. By providing a spacer between bone particles and active groups, the tether also reduces competition between chemical groups at the particle surface and the active group and makes the active group more accessible to monomers during polymerization.

In some embodiments, an active group is an analog of monomers of a polymer used in composite materials. For example, amine active groups will be incorporated into polyurethane matrices, copolymers (e.g., polyesters, polycarbonates, polycaprolactone), and other polymer classes based on monomers that react with amines, even if the polymer does not contain an amine. Hydroxy-terminated silanes will be incorporated into polyamino acids, polyesters, polycaprolactone, polycarbonates, polyurethanes, and other polymer classes that include hydroxylated monomers. Aromatic active groups or active groups with double bonds will be incorporated into vinyl polymers and other polymers that grow by radical polymerization (e.g., polyacrylates, polymethacrylates). It is not necessary that the active group be monofunctional. Indeed, it may be preferable that active groups that are to be incorporated into polymers via step polymerization be difunctional. A silane having two amines, even if one is a secondary amine, will not terminate a polymer chain but can react with ends of two different polymer chains. Alternatively, the active group may be branched to provide two reactive groups in the primary position.

An exemplary list of silanes that may be used with the present invention is provided in U.S. Patent Publication No. 2004/0146543, the contents of which are incorporated herein by reference. Silanes are available from companies such as Union Carbide, AP Resources Co. (Seoul, South Korea), and BASF. Where a silane contains a potentially non-biocompatible moiety as the active group, it may be used to tether a biocompatible compound to bone particles using a reaction in which the non-biocompatible moiety is a leaving group. It may be desirable to attach the biocompatible compound to the silane before attaching the silane to the bone particle, regardless of whether the silane is biocompatible or not. The derivatized silanes may be mixed with silanes that can be incorporated directly into the polymer and reacted with bone particles, coating the bone particles with a mixture of “bioactive” silanes and “monomer” silanes. U.S. Pat. No. 6,399,693, the contents of which are incorporated herein by reference discloses composite materials of silane modified polyaromatic polymers and bone. In some embodiments, silane-derivatized polymers may be used in composite materials instead of or in addition to first silanizing bone particles. In certain embodiments, polyurethanes and any copolymers used in accordance with the present inventions may not include silane modified polyaromatic polymers.

The active group of silanes may be incorporated directly into polymers or may be used to attach a second chemical group to bone particles. For example, if a particular monomer polymerizes through a functional group that is not commercially available as a silane, the monomer may be attached to the active group.

Non-silane linkers may also be employed to produce composite materials according to the invention. For example, isocyanates will form covalent bonds with hydroxyl groups on the surface of hydroxyapatite ceramics (de Wijn, et al., Fifth World Biomaterials Congress, May 29-Jun. 2, 1996, Toronto, CA). Isocyanate anchors, with tethers and active groups similar to those described with respect to silanes, may be used to attach monomer-analogs to bone particles or to attach chemical groups that will link covalently or non-covalently with a polymer side group. Polyamines, organic compounds containing one or more primary, secondary, or tertiary amines, will also bind with both the bone particle surface and many monomer and polymer side groups. Polyamines and isocyanates may be obtained from Aldrich.

Alternatively or additionally, biologically active compounds such as a biomolecule, a small molecule, or a bioactive agent may be attached to bone particles through a linker. For example, mercaptosilanes will react with sulfur atoms in proteins to attach them to bone particles. Aminated, hydroxylated, and carboxylated silanes will react with a wide variety functional groups. Of course, the linker may be optimized for the compound being attached to bone particles.

Biologically active molecules can modify non-mechanical properties of composite materials as they degrade. For example, immobilization of a drug on bone particles allows it to be gradually released at an implant site as a composite degrades. Anti-inflammatory agents embedded within composite materials will control inflammatory response long after an initial response to injection of the composite materials. For example, if a piece of the composite fractures several weeks after injection, immobilized compounds will reduce the intensity of any inflammatory response, and the composite will continue to degrade through hydrolytic or physiological processes. In some embodiments, compounds may also be immobilized on the bone particles that are designed to elicit a particular metabolic response or to attract cells to injection sites.

Some biomolecules, small molecules, and bioactive agents may also be incorporated into polyurethane matrices used in composite materials. For example, many amino acids have reactive side chains. The phenol group on tyrosine has been exploited to form polycarbonates, polyarylates, and polyiminocarbonates (see Pulapura, et al., Biopolymers, 1992, 32: 411-417; and Hooper, et al., J. Bioactive and Compatible Polymers, 1995, 10:327-340, the entire contents of both of which are incorporated herein by reference). Amino acids such as lysine, arginine, hydroxylysine, proline, and hydroxyproline also have reactive groups and are essentially tri-functional. Amino acids such as valine, which has an isopropyl side chain, are still difunctional. Such amino acids may be attached to the silane and still leave one or two active groups available for incorporation into a polymer.

Non-biologically active materials may also be attached to bone particles. For example, radiopaque (e.g., barium sulfate), luminescent (e.g., quantum dots), or magnetically active particles (e.g., iron oxide) may be attached to bone particles using the techniques described above. Mineralized bone particles are an inherently radiopaque component of some embodiments of present inventions, whereas demineralized bone particles, another optional component of composite materials, are not radiopaque. To enhance radiopacity of composite materials, mineralized bone particles can be used. Another way to render radiopaque the polymers utilized in accordance with the present inventions, is to chemically modify them such that a halogen (e.g., iodine) is chemically incorporated into the polyurethane matrices, as in U.S. patent application Ser. No. 10/952,202, now published as U.S. Patent Publication No. 2006-0034769, whose content is incorporated herein by reference.

If a material, for example, a metal atom or cluster, cannot be produced as a silane or other group that reacts with bone particles, then a chelating agent may be immobilized on bone particle surface and allowed to form a chelate with the atom or cluster. As bone particles and polymers used in the present invention are resorbed, these non-biodegradable materials may be removed from tissue sites by natural metabolic processes, allowing degradation of the polymers and resorption of the bone particles to be tracked using standard medical diagnostic techniques.

In some embodiments, bone particle surface is chemically treated before being mixed with polyurethane. For example, non-demineralized bone particles may be rinsed with phosphoric acid, e.g., for 1 to 15 minutes in a 5-50% solution by volume. Those skilled in the art will recognize that the relative volume of bone particles and phosphoric acid solution (or any other solution used to treat bone particles), may be optimized depending on the desired level of surface treatment. Agitation will also increase the uniformity of the treatment both along individual particles and across an entire sample of particles. A phosphoric acid solution reacts with mineral components of bone particles to coat the bone particles with calcium phosphate, which may increase the affinity of the surface for inorganic coupling agents such as silanes and for polymer components of the composite material. As noted above, bone particle surface may be partially demineralized to expose the collagen fibers.

Collagen fibers exposed by demineralization are typically relatively inert but have some exposed amino acid residues that can participate in reactions. Collagen may be rendered more reactive by fraying triple helical structures of the collagen to increase exposed surface area and number of exposed amino acid residues. This not only increases surface area of bone particles available for chemical reactions but also for their mechanical interactions with polymers as well. Rinsing partially demineralized bone particles in an alkaline solution will fray collagen fibrils. For example, bone particles may be suspended in water at a pH of about 10 for about 8 hours, after which the solution is neutralized. One skilled in the art will recognize that this time period may be increased or decreased to adjust the extent of fraying. Agitation, for example, in an ultrasonic bath, may reduce the processing time. Alternatively or additionally, bone particles may be sonicated with water, surfactant, alcohol, or some combination of these.

In some embodiments, collagen fibers at bone particle surface may be cross-linked. A variety of cross-linking techniques suitable for medical applications are well known in the art (see, for example, U.S. Pat. No. 6,123,781, the contents of which are incorporated herein by reference). For example, compounds like 1-ethyl-3-(3-dimethylaminopropyl) carbodiimide hydrochloride, either alone or in combination with N-hydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, may also be used to cross-link collagen chains (see Simmons, et al, Biotechnol. Appl. Biochem., 1993, 17:23-29; PCT Publication WO98/19718, the contents of both of which are incorporated herein by reference). Alternatively or additionally, hydroxymethyl phosphine groups on collagen may be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethylene diisocyanate, and sugars may also be used to cross-link collagens. Bone particles are then washed to remove all leachable traces of materials. In other embodiments, enzymatic cross-linking agents may be used. Additional cross-linking methods include chemical reaction, irradiation, application of heat, dehydrothermal treatment, enzymatic treatment, etc. One skilled in the art will easily be able to determine the optimal concentrations of cross-linking agents and incubation times for the desired degree of cross-linking.

Both frayed and unfrayed collagen fibers may be derivatized with monomer, pre-polymer, oligomer, polymer, initiator, and/or biologically active or inactive compounds, including but not limited to biomolecules, bioactive agents, small molecules, inorganic materials, minerals, through reactive amino acids on the collagen fiber such as lysine, arginine, hydroxylysine, proline, and hydroxyproline. Monomers that link via step polymerization may react with these amino acids via the same reactions through which they polymerize. Vinyl monomers and other monomers that polymerize by chain polymerization may react with these amino acids via their reactive pendant groups, leaving the vinyl group free to polymerize. Alternatively, or in addition, bone particles may be treated to induce calcium phosphate deposition and crystal formation on exposed collagen fibers. Calcium ions may be chelated by chemical moieties of the collagen fibers, and/or calcium ions may bind to the surface of the collagen fibers. James et al., Biomaterials 20:2203-2313, 1999; incorporated herein by reference. Calcium ions bound to the collagen provides a biocompatible surface, which allows for the attachment of cells as well as crystal growth. Polymer will interact with these fibers, increasing interfacial area and improving the wet strength of composite material.

In some embodiments, the surface treatments described above or treatments such as etching may be used to increase the surface area or surface roughness of bone particles. Such treatments increase the interfacial strength of the particle/polymer interface by increasing the surface area of the interface and/or the mechanical interlocking of bone particles and polyurethane. Such surface treatments may also be employed to round the shape or smooth the edges of bone particles to facilitate delivery of a composite material. Such treatment is particularly useful for injectable composite materials.

In some embodiments, surface treatments of bone particles are optimized to enhance covalent attractions between bone particles and polyurethanes. In some embodiments, the surface treatment may be designed to enhance non-covalent interactions between bone particle and polyurethane matrix. Exemplary non-covalent interactions include electrostatic interactions, hydrogen bonding, pi-bond interactions, hydrophobic interactions, van der Waals interactions, and mechanical interlocking. For example, if a protein or a polysaccharide is immobilized on bone particle, the chains of polymer matrix will become physically entangled with long chains of the biological macromolecules when they are combined. Charged phosphate sites on the surface of bone particles, produced by washing the bone particles in basic solution, will interact with the amino groups present in many biocompatible polymers, especially those based on amino acids. The pi-orbitals on aromatic groups immobilized on a bone particle will interact with double bonds and aromatic groups of the polymer.

Additional Particulate Materials. Any type of additional components comprising inorganic materials and/or other bone substitute materials (i.e., compositions similar to natural bone such as collagen, biocompatible polymers, osteoinductive agents, other commercial bone graft products, any composite graft, etc.), may be utilized in the present invention. Inorganic materials, including but not limited to, calcium phosphate materials, and other bone substitute materials, may also be exploited for use as particulate inclusions in composite materials used in the present invention. Exemplary materials utilized in accordance with the present invention include aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, calcium phosphate, hydroxyapatite, a-tricalcium phosphate, dicalcium phosphate, β-tricalcium phosphate, tetracalcium phosphate, amorphous calcium phosphate, octacalcium phosphate, and BIOGLASS™, a calcium phosphate silica glass available from U.S. Biomaterials Corporation. Substituted calcium phosphate phases are also contemplated for use with the invention, including but not limited to fluorapatite, chlorapatite, magnesium-substituted tricalcium phosphate, and carbonate hydroxyapatite. In certain embodiments, an inorganic material is a substituted form of hydroxyapatite. For example, hydroxyapatite may be substituted with other ions such as fluoride, chloride, magnesium, sodium, potassium, and groups such as silicates, silicon dioxides, carbonates, etc. Additional calcium phosphate phases suitable for use with the present invention include those disclosed in U.S. Pat. Nos. RE 33,161 and RE 33,221 to Brown et al.; 4,880,610; 5,034,059; 5,047,031; 5,053,212; 5,129,905; 5,336,264; and 6,002,065 to Constantz et al.; 5,149,368; 5,262,166 and 5,462,722 to Liu et al.; 5,525,148 and 5,542,973 to Chow et al., 5,717,006 and 6,001,394 to Daculsi et al., 5,605,713 to Boltong et al., 5,650,176 to Lee et al., and 6,206,957 to Driessens et al, and biologically-derived or biomimetic materials such as those identified in Lowenstam H A, Weiner S, On Biomineralization, Oxford University Press, 1989; each of which is incorporated herein by reference.

In some embodiments, a particular material (e.g., a particulate and a particular composite) is employed in composite materials (e.g., BRBC composite materials). In some embodiments, particular materials such as those described above and any combination thereof may be combined with proteins such as bovine serum albumin (BSA), collagen, or other extracellular matrix components to form a composite material. In some embodiments, a particular material is modified (e.g., surface modified) to be used in composite materials. In some embodiments, a particular material are combined with synthetic or natural polymers to form a composite material using the techniques described in co-pending U.S. patent applications, U.S. Ser. No. 10/735,135, filed Dec. 12, 2003; U.S. Ser. No. 10/681,651, filed Oct. 8, 2003; and U.S. Ser. No. 10/639,912, filed Aug. 12, 2003, the contents of all of which are incorporated herein by reference.

Polymer Component

A polymer component used in accordance with the present invention may include a polymer, a prepolymer, a monomer, or any combinations thereof.

Polymers useful for the preparation of composite materials (e.g., BRBC composite materials) include biocompatible polymers, that can be of natural or synthetic origin or a combination of natural and synthetic polymers. Biodegradable polymers may be used in some embodiments. Co-polymers and/or polymer blends may also be used in some embodiments. The polymers can be dendritic, branched, linear, substantially cross-linked, and/or substantially not cross-linked. A variety of polymers suitable for use in the present invention are known in the art, many of which are listed in commonly owned applications: U.S. application Ser. No. 10/735,135 filed on Dec. 12, 2003, entitled “Formable and settable polymer bone composite and method of production thereof” and published under No. 2005-0008672; U.S. application Ser. No. 10/681,651 filed on Oct. 8, 2003, entitled “Coupling agents for orthopedic biomaterials” and published under No. 2005-0008620; and U.S. Provisional Appln. No. 60/760,538, filed on Jan. 19, 2006 and entitled “Injectable and Settable Bone Substitute Material”, all of which are incorporated herein by reference.

A number of biodegradable and non-biodegradable biocompatible polymers suitable for use in the practice of the present invention are known in the field of polymeric biomaterials, controlled drug release, and tissue engineering (see, for example, U.S. Pat. Nos. 6,123,727; 5,804,178; 5,770,417; 5,736,372; and 5,716,404 to Vacanti; U.S. Pat. Nos. 6,095,148; and 5,837,752 to Shastri; U.S. Pat. No. 5,902,599 to Anseth; U.S. Pat. Nos. 5,696,175; 5,514,378; and 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; and 4,638,045 to Kohn; U.S. Pat. Appln. No. 2005-0013793 to Beckman; see also Langer, Acc. Chem. Res. 2000, 33: 94-101; Langer, J. Control Release, 1999, 62: 7-11; and Uhrich et al., Chem. Rev., 1999, 99: 3181-3198, the contents of all of which are incorporated herein by reference).

In some embodiments, the polymer is biodegradable. Exemplary biodegradable materials include lactide-glycolide copolymers of any ratio (e.g., 85:15, 40:60, 30:70, 25:75, or 20:80), poly(L-lactide-co-D,L-lactide), polyglyconate, poly(arylates), poly(anhydrides), poly(hydroxy acids), polyesters, poly(ortho esters), poly(alkylene oxides), polycarbonates, poly(propylene fumarates), poly(propylene glycol-co fumaric acid), poly(caprolactones), polyamides, polyamino acids, polyacetals, polylactides, polyglycolides, poly(dioxanones), polyhydroxybutyrate, polyhydroxyvalyrate, polyhydroxybutyrate/valerate copolymers, poly(vinyl pyrrolidone), biodegradable polycyanoacrylates, biodegradable polyurethanes including glucose-based polyurethanes and lysine-based polyurethanes, and polysaccharides (e.g., chitin, starches, celluloses). Natural polymers, including collagen, polysaccharides, agarose, glycosaminoglycans, alginate, chitin, and chitosan, may also be employed. Tyrosine-based polymers, including but not limited to polyarylates and polycarbonates, may also be employed (see Pulapura, et al., Biopolymers, 1992, 32: 411-417; Hooper, et al., J. Bioactive and Compatible Polymers, 1995, 10:327-340, the contents of both of which are incorporated herein by reference). Monomers for tyrosine-based polymers may be prepared by reacting an L-tyrosine-derived diphenol compound with phosgene or a diacid (Hooper, 1995; Pulapura, 1992). Similar techniques may be used to prepare amino acid-based monomers of other amino acids having reactive side chains, including imines, amines, thiols, etc. The polymers described in U.S. patent applications U.S. Ser. No. 11/336,127, filed on Jan. 19, 2006, and published under No. 2006-0216323, which is entitled “Polyurethanes for Osteoimplants”, and U.S. Ser. No. 10/771,736, filed on Feb. 4, 2004, and published under No. 2005-0027033, which is entitled “Polyurethanes for Osteoimplants”, may also be used in embodiments. In some embodiments, the degradation products include bioactive materials, biomolecules, small molecules, or other such materials that participate in biological processes.

Non-biodegradable polymers may also be used in the present invention. For example, polypyrrole, polyanilines, polythiophene, and derivatives thereof are useful electroactive polymers that can transmit voltage from endogenous bone to an implant. Other non-degradable, yet biocompatible polymers include polystyrene, polyesters, polyureas, poly(vinyl alcohol), polyamides, poly(tetrafluoroethylene), and expanded polytetrafluoroethylene (ePTFE), poly(ethylene vinyl acetate), polypropylene, polyacrylate, non-biodegradable polycyano-acrylates, non-biodegradable polyurethanes, mixtures and copolymers of poly(ethyl methacrylate) with tetrahydrofurfuryl methacrylate, polymethacrylate, poly(methyl methacrylate), polyethylene, including ultra high molecular weight polyethylene (UHMWPE), polypyrrole, polyanilines, polythiophene, poly(ethylene oxide), poly(ethylene oxide co-butylene terephthalate), poly ether-ether ketones (PEEK), and polyetherketoneketones (PEKK). Monomers that are used to produce any of these polymers are easily purchased from companies such as Polysciences, Sigma, and Scientific Polymer Products.

Examples of polymers for use with the present invention include but are not limited to starch-poly(caprolactone), poly(caprolactone), poly(lactide), poly(D,L-lactide), poly(lactide-co-glycolide), poly(D,L-lactide-co-glycolide), polycarbonates, polyurethane, tyrosine polycarbonate, tyrosine polyarylate, poly(orthoesters), polyphosphazenes, polypropylene fumarate, polyhydroxyvalerate, polyhydroxy butyrate, acrylates, methacrylates, and co-polymers, mixtures, enantiomers, and derivatives thereof. In certain particular embodiments, the polymer is starch-poly(caprolactone), poly(caprolactone), poly(lactide), poly(D,L-lactide), poly(lactide-co-glycolide), poly(D,L-lactide-co-glycolide), polyurethane, or a co-polymer, mixture, enantiomer, or derivative thereof. In certain embodiments, the polymer is poly(D,L-lactide). In certain other embodiments, the polymer is poly(D,L-lactide-co-glycolide). In certain embodiments, the polymer is poly(caprolactone). In certain embodiments, the polymer is a poly(urethane). In certain embodiments, the polymer is tyrosine polycarbonate. In certain embodiments, the polymer is tyrosine polyarylate.

In some embodiments, polymers used in composite materials (e.g., BRBC composite materials) utilized according to the present invention is poly(lactide-co-glycolide). The ratio of lactide and glycolide units in the polymer may vary. Particularly useful ratios are approximately 45-80% lactide to approximately 44-20% glycolide. In certain embodiments, the ratio is approximately 50% lactide to approximately 50% glycolide. In other certain embodiments, the ratio is approximately 65% lactide to approximately 45% glycolide. In other certain embodiments, the ratio is approximately 60% lactide to approximately 40% glycolide. In other certain embodiments, the ratio is approximately 70% lactide to approximately 30% glycolide. In other certain embodiments, the ratio is approximately 75% lactide to approximately 25% glycolide. In certain embodiments, the ratio is approximately 80% lactide to approximately 20% glycolide. In certain of the above embodiments, lactide is D,L-lactide. In other embodiments, lactide is L-lactide. In certain particular embodiments, RESOMER® 824 (poly-L-lactide-co-glycolide) (Boehringer Ingelheim) is used as polymer in a composite. In certain particular embodiments, RESOMER® 504 (poly-D,L-lactide-co-glycolide) (Boehringer Ingelheim) is used as polymer in a composite. In certain particular embodiments, PURASORB PLG (75/25 poly-L-lactide-co-glycolide) (Purac Biochem) is used as polymer in a composite. In certain particular embodiments, PURASORB PG (polyglycolide) (Purac Biochem) is used as polymer in a composite. In certain embodiments, a polymer is PEGylated-poly(lactide-co-glycolide). In certain embodiments, a polymer is PEGylated-poly(lactide). In certain embodiments, a polymer is PEGylated-poly(glycolide). In other embodiments, a polymer is polyurethane. In other embodiments, a polymer is polycaprolactone. In certain embodiments, a polymer is a copolymer of poly(caprolactone) and poly(lactide).

For polyesters such as poly(lactide) and poly(lactide-co-glycolide), the inherent viscosity of a polymer ranges from about 0.4 dL/g to about 5 dL/g. In certain embodiments, the inherent viscosity of a polymer ranges from about 0.6 dL/g to about 2 dL/g. In certain embodiments, the inherent viscosity of a polymer ranges from about 0.6 dL/g to about 3 dL/g. In certain embodiments, the inherent viscosity of a polymer ranges from about 1 dL/g to about 3 dL/g. In certain embodiments, the inherent viscosity of a polymer ranges from about 0.4 dL/g to about 1 dL/g. For poly(caprolactone), the inherent viscosity of the polymer ranges from about 0.5 dL/g to about 1.5 dL/g. In certain embodiments, the inherent viscosity of the poly(caprolactone) ranges from about 1.0 dL/g to about 1.5 dL/g. In certain embodiments, the inherent viscosity of the poly(caprolactone) ranges from about 1.0 dL/g to about 1.2 dL/g. In certain embodiments, the inherent viscosity of the poly(caprolactone) is about 1.08 dL/g.

Those skilled in the art will recognize that this an exemplary, not a comprehensive, list of polymers appropriate for in vivo applications. Co-polymers, mixtures, and adducts of the above polymers may also be used in the practice of the present invention.

Polyurethane According to the present invention, polyurethanes (PUR) are a useful class of biomaterials to be included in composite materials (e.g., BRBC composite materials) for use in accordance with the present invention due to the fact that they can be injectable or moldable as a reactive liquid that subsequently cures to form a composite when the composite material sets or hardens. Polyurethanes also have tunable degradation rates, which are shown to be highly dependent on the choice of polyol and isocyanate components (Hafeman et al., Pharmaceutical Research 2008; 25(10):2387-99; Storey et al., J Poly Sci Pt A: Poly Chem 1994; 32:2345-63; Skarja et al., J App Poly Sci 2000; 75:1522-34). Polyurethanes have tunable mechanical properties, which can also be enhanced with the addition of bone particles and/or other components (Adhikari et al., Biomaterials 2008; 29:3762-70; Gorna et al., J Biomed Mater Res Pt A 2003; 67A(3):813-27) and exhibit elastomeric rather than brittle mechanical properties.

Polyurethanes can be made by reacting together the components of a two-component composition, one of which includes a polyisocyanate while the other includes a component having two or more hydroxyl groups (i.e., polyols) to react with a polyisocyanate. For example, U.S. Pat. No. 6,306,177, discloses a method for repairing a tissue site using polyurethanes, the content of which is incorporated by reference.

It is to be understood that by “a two-component composition” it means a composition comprising two essential types of polymer components. In some embodiments, such a composition may additionally comprise one or more other optional components.

In some embodiments, polyurethane is a polymer that has been rendered formable through combination of two liquid components (i.e., a polyisocyanate prepolymer and a polyol). In some embodiments, a polyisocyanate prepolymer or a polyol may be a molecule with two or three isocyanate or hydroxyl groups respectively. In some embodiments, a polyisocyanate prepolymer or a polyol may have at least four isocyanate or hydroxyl groups respectively.

Synthesis of polyurethane results from a balance of two simultaneous reactions. Reactions, in some embodiments, are illustrated below in Scheme 1. One is a gelling reaction, where an isocyanates and a polyester polyol react to form urethane bonds. The one is a blowing reaction. An isocyanate can react with water to form carbon dioxide gas, which acts as a lowing agent to form pores of polyurethane foam. The relative rates of these reactions determine the scaffold morphology, working time, and setting time.

Exemplary gelling and blowing reactions in forming of polyurethane are shown in Scheme 1 below, where R₁, R₂ and R₃, for example, can be oligomers of caprolactone, lactide and glycolide respectively.

### Gelling Reaction

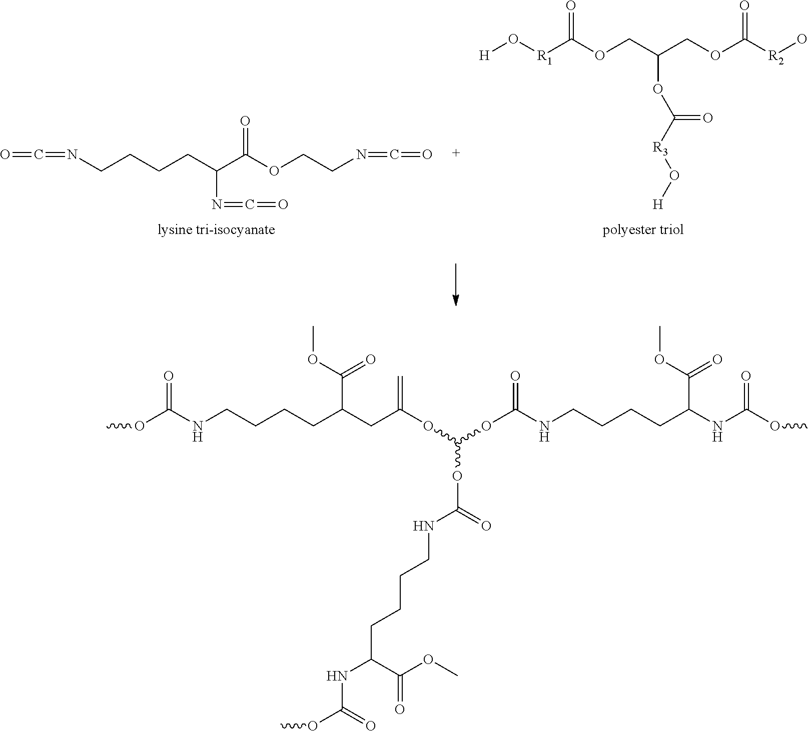

### Blowing Reaction

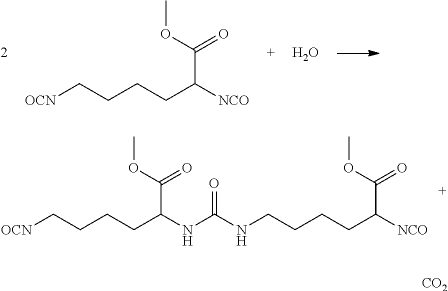

Biodegradable polyurethane scaffolds synthesized from aliphatic polyisocyanates been shown to degrade into non-toxic compounds and support cell attachment and proliferation in vitro. A variety of polyurethane polymers suitable for use in the present invention are known in the art, many of which are listed in commonly owned applications: U.S. Ser. No. 10/759,904 filed on Jan. 16, 2004, entitled “Biodegradable polyurethanes and use thereof” and published under No. 2005-0013793; U.S. Ser. No. 11/667,090 filed on Nov. 5, 2005, entitled “Degradable polyurethane foams” and published under No. 2007-0299151; U.S. Ser. No. 12/298,158 filed on Apr. 24, 2006, entitled “Biodegradable polyurethanes” and published under No. 2009-0221784; all of which are incorporated herein by reference. Polyurethanes described in U.S. Ser. No. 11/336,127 filed on Jan. 19, 2006 and published under No. 2006-0216323, which is entitled “Polyurethanes for Osteoimplants” and incorporated herein by reference, may be used in some embodiments of the present invention.

Polyurethanes-based composite materials (e.g, BRBC composite materials) may be prepared by contacting an isocyanate-terminated prepolymer (component 1, e.g, polyisocyanate prepolymer) with a hardener (component 2) that includes at least a polyol (e.g., a polyester polyol) and water, a catalyst and optionally, a stabilizer, a porogen, PEG, etc. In some embodiments, multiple polyurethanes (e.g., different structures, difference molecular weights) may be used in a composite material (e.g., BRBC composite materials) of the present invention. In some embodiments, other biocompatible and/or biodegradable polymers may be used with polyurethanes in accordance with the present invention. In some embodiments, biocompatible co-polymers and/or polymer blends of any combination thereof may be exploited.

Composite materials (e.g, BRBC composite materials) including polyurethanes as a polymer component used in accordance with the present invention can be adjusted to produce polymers having various physiochemical properties and morphologies including, for example, flexible foams, rigid foams, elastomers, coatings, adhesives, and sealants. The properties of polyurethanes are controlled by choice of the raw materials and their relative concentrations. For example, thermoplastic elastomers are characterized by a low degree of cross-linking and are typically segmented polymers, consisting of alternating hard (diisocyanates and chain extenders) and soft (polyols) segments. Thermoplastic elastomers are formed from the reaction of diisocyanates with long-chain diols and short-chain diol or diamine chain extenders. In some embodiments, pores in bone/polyurethanes composite materials in the present invention are interconnected and have a diameter ranging from approximately 50 to approximately 1000 microns.

Prepolymer. Polyurethane prepolymers can be prepared by contacting a polyol with an excess (typically a large excess) of a polyisocyanate. The resulting prepolymer intermediate includes an adduct of polyisocyanates and polyols solubilized in an excess of polyisocyanates. Prepolymer can, in some embodiments, be formed by using an approximately stoichiometric amount of polyisocyanates in forming a prepolymer and subsequently adding additional polyisocyanates. The prepolymer therefore exhibits both low viscosity, which facilitates processing, and improved miscibility as a result of the polyisocyanate-polyol adduct. Polyurethane networks can, for example, then be prepared by reactive liquid molding, wherein the prepolymer is contacted with a polyester polyol to form a reactive liquid mixture (i.e., a two-component composition) which is then cast into a mold and cured.

Polyisocyanates or multi-isocyanate compounds for use to make BRBC composite materials include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. In some embodiments, hexamethylene diisocyanate (HDI) trimer sold as Desmodur N3300A may be a polyisocyanate utilized in the present invention. In some embodiments, polyisocyanates used in the present invention includes approximately 10 to 55% NCO by weight (wt % NCO=100*(42/Mw)). In some embodiments, polyisocyanates include approximately 15 to 50% NCO.

Polyisocyanate prepolymers provide an additional degree of control over the structure of biodegradable polyurethanes. Prepared by reacting polyols with isocyanates, NCO-terminated prepolymers are oligomeric intermediates with isocyanate functionality as shown in Scheme 1. To increase reaction rates, urethane catalysts (e.g., tertiary amines) and/or elevated temperatures (60-90° C.) may be used (see, Guelcher, Tissue Engineering: Part B, 14 (1) 2008, pp 3-17).

Polyols used to react with polyisocyanates in preparation of NCO-terminated prepolymers refer to molecules having at least two functional groups to react with isocyanate groups. In some embodiments, polyols have a molecular weight of no more than 1000 g/mol. In some embodiments, polyols have a rang of molecular weight between about 100 g/mol to about 500 g/mol. In some embodiments, polyols have a rang of molecular weight between about 200 g/mol to about 400 g/mol. In certain embodiments, polyols (e.g., PEG) have a molecular weight of about 200 g/mol. Exemplary polyols include, but are not limited to, PEG, glycerol, pentaerythritol, dipentaerythritol, tripentaerythritol, 1,2,4-butanetriol, trimethylolpropane, 1,2,3-trihydroxyhexane, myo-inositol, ascorbic acid, a saccharide, or sugar alcohols (e.g., mannitol, xylitol, sorbitol etc.). In some embodiments, polyols may comprise multiple chemical entities having reactive hydrogen functional groups (e.g., hydroxy groups, primary amine groups and/or secondary amine groups) to react with the isocyanate functionality of polyisocyanates.

In some embodiments, polyisocyanate prepolymers are resorbable. Zhang and coworkers synthesized biodegradable lysine diisocyanate ethyl ester (LDI)/glucose polyurethane foams proposed for tissue engineering applications. In those studies, NCO-terminated prepolymers were prepared from LDI and glucose. The prepolymers were chain-extended with water to yield biocompatible foams which supported the growth of rabbit bone marrow stromal cells in vitro and were non-immunogenic in vivo. (see Zhang, et al., Biomaterials 21: 1247-1258 (2000), and Zhang, et al., Tiss. Eng., 8(5): 771-785 (2002), both of which are incorporated herein by reference).

In some embodiments, prepared polyisocyanate prepolymer can be a flowable liquid at processing conditions. In general, the processing temperature is no greater than 60° C. In some embodiments, the processing temperature is ambient temperature (25° C.).

Polyols. Polyols utilized in accordance with the present invention can be amine- and/or hydroxyl-terminated compounds and include, but are not limited to, polyether polyols (such as polyethylene glycol (PEG or PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). In some embodiments, polyols may be (1) molecules having multiple hydroxyl or amine functionality, such as glucose, polysaccharides, and castor oil; and (2) molecules (such as fatty acids, triglycerides, and phospholipids) that have been hydroxylated by known chemical synthesis techniques to yield polyols.

Polyols used in the present invention may be polyester polyols. In some embodiments, polyester polyols include polyalkylene glycol esters or polyesters prepared from cyclic esters. In some embodiments, polyester polyols include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. In some embodiments, polyester polyols include, polyesters prepared from caprolactone, glycolide, D, L-lactide, mixtures thereof, and/or copolymers thereof. In some embodiments, polyester polyols, for example, include polyesters prepared from castor-oil. When polyurethanes degrade, their degradation products can be the polyols from which they were prepared from.

In some embodiments, polyester polyols are miscible with prepared prepolymers used in reactive liquid mixtures (i.e., two-component composition) of the present invention. In some embodiments, surfactants or other additives are included in the reactive liquid mixtures to help homogenous mixing.

The glass transition temperature (Tg) of polyester polyols used in the reactive liquids to form polyurethanes can be less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

Molecular weight of polyester polyols used in the reactive liquids to form polyurethanes can, for example, be adjusted to control the mechanical properties of polyurethanes utilized in accordance with the present invention. In that regard, using polyester polyols of higher molecular weight results in greater compliance or elasticity. In some embodiments, polyester polyols used in the reactive liquids may have a molecular weight less than approximately 3000 Da. In certain embodiments, the molecular weight may be in the range of approximately 200 to 2500 Da or 300 to 2000 Da. In some embodiments, the molecular weight may be approximately in the range of approximately 450 to 1800 Da or 450 to 1200 Da.

In some embodiments, a polyester polyol comprise poly(caprolactone-co-lactide-co-glycolide), which has a molecular weight in a range of 200 Da to 2500 Da, or 300 Da to 2000 Da.

In some embodiments, polyols may include multiply types of polyols with different structures, molecular weight, properties, etc.

Additional Components. In accordance with the present invention, two-component compositions (i.e., polyprepolymers and polyols) to form porous composite materials may be used with other agents and/or catalysts. Zhang et al. have found that water may be an adequate blowing agent for a lysine diisocyanate/PEG/glycerol polyurethane (see Zhang, et al., Tissue Eng. 2003 (6):1143-57) and may also be used to form porous structures in polyurethanes. Other blowing agents include dry ice or other agents that release carbon dioxide or other gases into the composite. Alternatively, or in addition, porogens (see detail discussion below) such as salts may be mixed in with reagents and then dissolved after polymerization to leave behind small voids.

Two-component compositions of polyurethanes and/or the prepared composite materials (e.g, BRBC composite materials) used in the present invention may include one or more additional components. In some embodiments, compositions and/or composite materials may includes, water, a catalyst (e.g., gelling catalyst, blowing catalyst, etc.), a stabilizer, a plasticizer, a porogen, a chain extender (for making of polyurethanes), a pore opener (such as calcium stearate, to control pore morphology), a wetting or lubricating agent, etc. (See, U.S. Ser. No. 10/759,904 published under No. 2005-0013793, and U.S. Ser. No. 11/625,119 published under No. 2007-0191963; both of which are incorporated herein by reference).

In some embodiments, composite materials (e.g, BRBC composite materials) include and/or be combined with a solid filler (e.g., carboxymethylcellulose (CMC), hyaluronic acid (HA) and PMMA). For example, when composite materials used in wound healing, solid fillers can help absorb excess moisture in the wounds from blood and serum and allow for proper foaming.

In certain embodiments, additional biocompatible polymers (e.g., PEG) or co-polymers can be used with compositions and composite materials in the present invention.

Water may be a blowing agent to generate porous polyurethane-based composite materials (e.g, BRBC composite materials). Porosity of a composite material (e.g., when hardened) increased with increasing water content, and biodegradation rate accelerated with decreasing polyester half-life, thereby yielding a family of materials with tunable properties that are useful in the present invention. See, Guelcher et al., Tissue Engineering, 13(9), 2007, pp 2321-2333, which is incorporated by reference.

In some embodiments, an amount of water is about 0.5, 1, 1.5, 2, 3, 4, 5, 6, 7, 8, 9, 10 parts per hundred parts (pphp) polyol. In some embodiments, water has an approximate rang of any of such amounts.

In some embodiments, at least one catalyst is added to form reactive liquid mixture (i.e., two-component compositions). A catalyst, for example, can be non-toxic (in a concentration that may remain in the polymer).

A catalyst can, for example, be present in two-component compositions in a concentration in the range of approximately 0.5 to 5 parts per hundred parts polyol (pphp) and, for example, in the range of approximately 0.5 to 2, or 2 to 3 pphp. A catalyst can, for example, be an amine compound. In some embodiments, catalyst may be an organometallic compound or a tertiary amine compound. In some embodiments the catalyst may be stannous octoate (an organobismuth compound), triethylene diamine, bis(dimethylaminoethyl)ether, dimethylethanolamine, dibutyltin dilaurate, and Co scat organometallic catalysts manufactured by Vertullus (a bismuth based catalyst), or any combination thereof.

In some embodiments, a stabilizer is nontoxic (in a concentration remaining in the polyurethane foam) and can include a non-ionic surfactant, an anionic surfactant or combinations thereof. For example, a stabilizer can be a polyethersiloxane, a salt of a fatty sulfonic acid or a salt of a fatty acid. In certain embodiments, a stabilizer is a polyethersiloxane, and concentration of polyethersiloxane in a reactive liquid mixture can, for example, be in the range of approximately 0.25 to 4 parts per hundred polyol. In some embodiments, polyethersiloxane stabilizer are hydrolyzable.

In some embodiments, the stabilizer can be a salt of a fatty sulfonic acid. Concentration of a salt of the fatty sulfonic acid in a reactive liquid mixture can be in the range of approximately 0.5 to 5 parts per hundred polyol. Examples of suitable stabilizers include a sulfated castor oil or sodium ricinoleicsulfonate.

Stabilizers can be added to a reactive liquid mixture of the present invention to, for example, disperse prepolymers, polyols and other additional components, stabilize the rising carbon dioxide bubbles, and/or control pore sizes of composite materials. Without limitation to any mechanism of operation, it is believed that stabilizers preserve thermodynamically unstable state of a polyurethane-based composite materials (e.g, BRBC composite materials) during the time of rising by surface forces until composite materials (e.g, BRBC composite materials) is hardened. In that regard, stabilizers lower the surface tension of the mixture of starting materials and operate as emulsifiers for the system. Stabilizers, catalysts and other polyurethane reaction components are discussed, for example, in Oertel, Günter, ed., Polyurethane Handbook, Hanser Gardner Publications, Inc. Cincinnati, Ohio, 99-108 (1994). A specific effect of stabilizers is believed to be the formation of surfactant monolayers at interface of higher viscosity of bulk phase, thereby increasing elasticity of surface and stabilizing expanding foam bubbles.

To prepare high-molecular-weight polymers, in some embodiments, prepolymers are chain extended by adding a short-chain (e.g., <500 g/mol) polyamine or polyol. In certain embodiments, water may act as a chain extender. In some embodiments, addition of chain extenders with a functionality of two (e.g., diols and diamines) yields linear alternating block copolymers.

In some embodiments, composite materials (e.g, BRBC composite materials) include one or more plasticizers. Plasticizers are typically compounds added to polymers or plastics to soften them or make them more pliable. According to the present invention, plasticizers soften, make workable, or otherwise improve the handling properties of polymers or composite materials (e.g, BRBC composite materials). Plasticizers also allow composite materials to be moldable at a lower temperature (e.g., room temperature), thereby avoiding heat induced tissue necrosis during implantation. Plasticizer may evaporate or otherwise diffuse out of composite materials over time, thereby allowing composite materials to harden or set. Without being bound to any theory, plasticizer are thought to work by embedding themselves between the chains of polymers. This forces polymer chains apart and thus lowers glass transition temperature of polymers. In general, the more plasticizer added, the more flexible the resulting polymers or composites will be.

In some embodiments, plasticizers are based on an ester of a polycarboxylic acid with linear or branched aliphatic alcohols of moderate chain length. For example, some plasticizers are adipate-based. Examples of adipate-based plasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl) phthalate (DEHP), diisononyl phthalate (DINP), bis(n-butyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid polyhydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl) trimellitate (TEHTM-MG), tri-(n-octyl,n-decyl) trimellitate (ATM), tri-(heptyl,nonyl) trimellitate (LTM), n-octyl trimellitate (OTM)), benzoates, epoxidized vegetable oils, sulfonamides (e.g., N-ethyl toluene sulfonamide (ETSA), N-(2-hydroxypropyl) benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to BRBC composite materials as plasticizers. In certain particular embodiments, polymers with the same chemical structure as those used in BRBC composite materials are used but with lower molecular weights to soften overall composites. In other embodiments, different polymers with lower melting points and/or lower viscosities than those of the polymer component of BRBC composite materials are used.

In some embodiments, polymers used as plasticizer are poly(ethylene glycol) (PEG). PEG used as a plasticizer is typically a low molecular weight PEG such as those having an average molecular weight of 1000 to 10000 g/mol, for example, from 4000 to 8000 g/mol. In certain embodiments, PEG 4000, PEG 5000, PEG 6000, PEG 7000, PEG 8000 or combinations thereof are used in composite materials (e.g, BRBC composite materials). For example, plasticizer (PEG) is useful in making more moldable composite materials that include poly(lactide), poly(D,L-lactide), poly(lactide-co-glycolide), poly(D,L-lactide-co-glycolide), or poly(caprolactone). Plasticizer may comprise 1-40% of a composite material by weight. In some embodiments, a plasticizer is 10-30% by weight. In some embodiments, a plasticizer is approximately 10%, 15%, 20%, 25%, 30% or 40% by weight. In other embodiments, a plasticizer is not used in BRBC composite materials. For example, in some polycaprolactone-containing BRBC, a plasticizer is not used.

In some embodiments, inert plasticizers may be used. In some embodiments, a plasticizer may not be used in the present invention.

Porosity of a hardened composite materials (e.g, BRBC composite materials) may be accomplished using any means known in the art. Exemplary methods of creating porosity in a composite include, but are not limited to, particular leaching processes, gas foaming processing, supercritical carbon dioxide processing, sintering, phase transformation, freeze-drying, cross-linking, molding, porogen melting, polymerization, melt-blowing, and salt fusion (Murphy et al., Tissue Engineering 8(1):43-52, 2002; incorporated herein by reference). For a review, see Karageorgiou et al., Biomaterials 26:5474-5491, 2005; incorporated herein by reference. Porosity may be a feature of a gradually hardened composite material (e.g, a BRBC composite material) during or after surgeries (e.g., TKA).

Porogens may be any chemical compound that will reserve a space within a composite material while a composite material is being molded and will diffuse, dissolve, and/or degrade prior to or after implantation or injection leaving a pore in a composite material. Porogens may have the property of not being appreciably changed in shape and/or size during the procedure to make a composite material moldable. For example, a porogen should retain its shape during the heating of composite materials (e.g, BRBC composite materials) to make it moldable. Therefore, a porogen does not melt upon heating of a BRBC composite material to make it moldable. In certain embodiments, a porogen has a melting point greater than about 60° C., greater than about 70° C., greater than about 80° C., greater than about 85° C., or greater than about 90° C.

Porogens may be of any shape or size. A porogen may be spheroidal, cuboidal, rectangular, elonganted, tubular, fibrous, disc-shaped, platelet-shaped, polygonal, etc. In certain embodiments, a porogen is granular with a diameter ranging from approximately 100 microns to approximately 800 microns. In certain embodiments, a porogen is elongated, tubular, or fibrous. Such porogens provide increased connectivity of pores of composite material and/or also allow for a lesser percentage of the porogen in the composite.

Amount of porogens may vary in composites (e.g, BRBC composite materials) from 1% to 80% by weight. In certain embodiments, a plasticizer makes up from about 5% to about 80% by weight of the composite. In certain embodiments, a plasticizer makes up from about 10% to about 50% by weight of a composite material. Pores in composite materials when hardened are thought to improve the osteoinductivity or osteoconductivity of the composite by providing holes for cells such as osteoblasts, osteoclasts, fibroblasts, cells of osteoblast lineage, stem cells, etc. Pores provide composite materials with biological in growth capacity. Pores may also provide for easier degradation of composites as bone is formed and/or remodeled. In some embodiments, a porogen is biocompatible.

A porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the osteoimplant before or after implantation thereby providing pores for biological in-growth. Solid porogens may be crystalline or amorphous. Examples of possible solid porogens include water soluble compounds. Exemplary porogens include carbohydrates (e.g., sorbitol, dextran (poly(dextrose)), starch), salts, sugar alcohols, natural polymers, synthetic polymers, and small molecules.

In some embodiments, carbohydrates are used as porogens in composite materials (e.g, BRBC composite materials). A carbohydrate may be a monosaccharide, disaccharide, or polysaccharide. The carbohydrate may be a natural or synthetic carbohydrate. In some embodiments, a carbohydrate is a biocompatible, biodegradable carbohydrate. In certain embodiments, the carbohydrate is a polysaccharide. Exemplary polysaccharides include cellulose, starch, amylose, dextran, poly(dextrose), glycogen, etc.

In certain embodiments, a polysaccharide is dextran. Very high molecular weight dextran has been found particularly useful as a porogen. For example, the molecular weight of the dextran may range from about 500,000 g/mol to about 10,000,000 g/mol, preferably from about 1,000,000 g/mol to about 3,000,000 g/mol. In certain embodiments, the dextran has a molecular weight of approximately 2,000,000 g/mol. Dextrans with a molecular weight higher than 10,000,000 g/mol may also be used as porogens. Dextran may be used in any form (e.g., particles, granules, fibers, elongated fibers) as a porogen. In certain embodiments, fibers or elongated fibers of dextran are used as a porogen in composite materials. Fibers of dextran may be formed using any known method including extrusion and precipitation. Fibers may be prepared by precipitation by adding an aqueous solution of dextran (e.g., 5-25% dextran) to a less polar solvent such as a 90-100% alcohol (e.g., ethanol) solution. The dextran precipitates out in fibers that are particularly useful as porogens in composite materials (e.g, BRBC composite materials). Once composite materials with dextran as a porogen is applied to a implant site, dextran dissolves away very quickly. Within approximately 24 hours, substantially all of dextran is out of a hardened composite material leaving behind pores in it. An advantage of using dextran is that dextran exhibits a hemostatic property in extravascular space. Therefore, dextran in a composite material can decrease bleeding at or near surgical sites.

Small molecules including pharmaceutical agents may also be used as porogens in composite materials. Examples of polymers that may be used as plasticizers include poly(vinyl pyrollidone), pullulan, poly(glycolide), poly(lactide), and poly(lactide-co-glycolide). Typically low molecular weight polymers are used as porogens. In certain embodiments, a porogen is poly(vinyl pyrrolidone) or a derivative thereof. Plasticizers that are removed faster than the surrounding composite can also be considered porogens.

Components to be Delivered

Alternatively or additionally, composite materials (e.g, BRBC composite materials) utilized in accordance with the present invention may have one or more components to deliver when implanted, including biomolecules, small molecules, bioactive agents, etc., to promote bone growth and connective tissue regeneration, and/or to accelerate healing. Examples of materials that can be incorporated include chemotactic factors, angiogenic factors, bone cell inducers and stimulators, including the general class of cytokines such as the TGF-β superfamily of bone growth factors, the family of bone morphogenic proteins, osteoinductors, and/or bone marrow or bone forming precursor cells, isolated using standard techniques. Sources and amounts of such materials that can be included are known to those skilled in the art.

Biologically active materials, comprising biomolecules, small molecules, and bioactive agents may also be included in composite materials (e.g, BRBC composite materials) to, for example, stimulate particular metabolic functions, recruit cells, or reduce inflammation. For example, nucleic acid vectors, including plasmids and viral vectors, that will be introduced into the patient's cells and cause the production of growth factors such as bone morphogenetic proteins may be included in a composite material. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, hydroxyapatite, and penetraction enhancer. Additional exemplary substances include chemotactic factors, angiogenic factors, analgesics, antibiotics, anti-inflammatory agents, bone morphogenic proteins, and other growth factors that promote cell-directed degradation or remodeling of the polymer phase of the composite material and/or development of new tissue (e.g., bone). RNAi or other technologies may also be used to reduce the production of various factors.

In some embodiments, composite materials (e.g, BRBC composite materials) incorporate antibiotics. Antibiotics may be bacteriocidial or bacteriostatic. An anti-microbial agent may be included in composite materials. For example, anti-viral agents, anti-protazoal agents, anti-parasitic agents, etc. may be include in composite materials. Other suitable biostatic/biocidal agents include antibiotics, povidone, sugars, and mixtures thereof. Exemplary antibiotics include, but not limit to, Amikacin, Gentamicin, Kanamycin, Neomycin, Netilmicin, Streptomycin, Tobramycin, Paromomycin, Geldanamycin, Herbimycin, Loravabef, etc. (See, The Merck Manual of Medical Information—Home Edition, 1999).

Composite materials (e.g, BRBC composite materials) may also be seeded with cells. In some embodiments, a patient's own cells are obtained and used in such composite materials. Certain types of cells (e.g., osteoblasts, fibroblasts, stem cells, cells of the osteoblast lineage, etc.) may be selected for use in a composite material. Cells may be harvested from marrow, blood, fat, bone, muscle, connective tissue, skin, or other tissues or organs. In some embodiments, a patient's own cells may be harvested, optionally selected, expanded, and used in a composite material. In other embodiments, a patient's cells may be harvested, selected without expansion, and used in a composite material. Alternatively, exogenous cells may be employed. Exemplary cells for use with the invention include mesenchymal stem cells and connective tissue cells, including osteoblasts, osteoclasts, fibroblasts, preosteoblasts, and partially differentiated cells of the osteoblast lineage. Cells may be genetically engineered. For example, cells may be engineered to produce a bone morphogenic protein.

In some embodiments, a composite materials (e.g, BRBC composite materials) utilized in the present application may include a composite comprising a component to be delivered. For example, a composite can be a biomolecule (e.g., a protein) encapsulated in a polymeric microsphere or nanoparticles. In certain embodiments, BMP-2 encapsulated in PLGA microspheres may be embedded in a bone/polyurethane composite material used in accordance with the present invention. Sustained release of BMP-2 can be achieved due to the diffusional barriers presented by both PLGA and polyurethane of the composite. Thus, release kinetics of growth factors (e.g., BMP-2) can be tuned by varying size of PLGA micro spheres and porosity of polyurethane composite.

To enhance biodegradation in vivo, composite materials (e.g, BRBC composite materials) utilized in accordance with the present invention can also include different enzymes. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to ensure sufficient bone ingrowth while degrading.

Components to be delivered may not be covalently bonded to a component of composite materials utilized in the present application. In some embodiments, components may be selectively distributed on or near the surface of hardened composite materials (e.g, BRBC composite materials) at bone-implant interfaces using techniques such as layering as well know in the art. Alternatively or in addition, biologically active components may be covalently linked to bone particles before combination with a polymer. As discussed above, for example, silane coupling agents having amine, carboxyl, hydroxyl, or mercapto groups may be attached to the bone particles through the silane and then to reactive groups on a biomolecule, small molecule, or bioactive agent.

Composite Materials and Properties

Composite materials (i.e., BRBC composite materials) for use as described herein are unified combinations of particulate (e.g., bone particle) and polymeric (e.g., polyurethane) materials, optionally with one or more additional materials.

Such composite materials (i.e., BRBC composite materials) may include practically any ratio of polyurethane and bone particles. For example, in some embodiments, composite materials include between about 5 wt % and about 95 wt % particles. In some embodiments, composite materials include about 40 wt % to about 45 wt % particles, about 45 wt % to about 50 wt % particles or about 50 wt % to about 55 wt % particles. In some embodiments, composite materials include about 55 wt % to about 70 wt % particles. In some embodiments, composite materials include about 70 wt % to about 90 wt % particles. In some embodiments, composite materials include at least approximately 40 wt %, 45 wt %, 50 wt %, or 55 wt % of particles. In some embodiments, a weight percentage of particles in composite materials in accordance with the present invention is about 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt % or between any weight percentages of above. In some embodiments, as described herein, such particles are or comprise bone particles. In some embodiments, such particles include one or more non-bone materials (e.g., a calcium phosphate material).

In some embodiments, composite materials include at most approximately 30 vol %, 35 vol %, 40 vol %, or 50 vol % particles. In some embodiments, a volume percentage of particles in composite materials in accordance with the present invention may be about 10 vol %, 15 vol %, 20 vol %, 25 vol %, 30 vol %, 35 vol %, 40 vol %, 45 vol %, 50 vol %, 55 vol %, 60 vol %, 65 vol %, 70 vol %, 75 vol % or between any volume percentages of above. In some embodiments, composite materials in accordance with the present invention have a volume percentage (fraction) of at most approximately 36 vol % of particles. In some embodiments, volume percentages (fractions) of particles is less than 64 vol %. In some embodiments, volume percentages (fractions) of particles is the range of 50-60 vol %.

In certain embodiments, for a certain volume percentage, corresponding weight percentage of particulate materials is determined by density of the particulate materials. Furthermore, in some embodiments, where two or more different particulate materials are utilized, relative proportions of individual components of particulate materials are determined at least in part by relative densities.

Those of ordinary skill in the art, reading the present disclosure, will readily appreciate that identity, structure, and/or relative amounts of individual polymer and/or particulate material components may be selected upon consideration of any of a variety of factors including, for example, nature of the target sites (e.g., implant sites), shape and size of particles to be utilized, how evenly polymer and particles are distributed, desired flowability of composite materials, desired working time (e.g., 8-15 minutes), desired degree of moldability, desired mechanical properties of composite materials (e.g., degree of strength, degree of hardness, time period over which a particular strength (e.g., compressive strength, compressive modulus, shear modulus, isotropic ultimate shear stress, ultimate torsional stress, fatigue strength, etc.) or hardness is achieved, length of time over which a particular strength or hardness is maintained, etc), desired degradation properties of composite materials, desired rate of cellular infiltration, desired rate of remodeling, etc.

For example, as described herein, increasing proportions of particulate materials as compared with polymer materials often will increase viscosity of composite materials. Increased viscosity can alter ability of users to inject or mold a composite material.

Alternatively or additionally, use of particulate materials with a large size distribution may provide different characteristics than are achieved with particulate materials of more consistent size.

In general, it will typically be the case that use of lower molecular weight polymer materials (e.g., polyols) will impart slower degradation, and may also decrease certain mechanical properties such as strength. Adjusting porosity can also alter degradation rate and/or mechanical properties such as strength.

As described herein, composite materials (e.g., BRBC composite materials) are initially prepared at a first time, and then harden over time. During such hardening, certain mechanical and/or physical properties of composite materials (e.g., porosity, strength, flowability, degradation rate, etc) change. For example, composite materials (e.g., BRBC composite materials) may be provided in a flowable state with an initial flowability when applied to bone and implant components, and then set into a hardened state with less flowability than the initial flowability.

In some embodiments, composite materials (e.g., BRBC composite materials) have a porosity of more than 2 vol % or less than 15 vol % before being hardened. In some embodiments, a porosity of composite materials is 5 vol %, 10 vol %, 15 vol %, 20 vol %, 25 vol %, 30 vol %, 35 vol %, 40 vol %, 45 vol %, 50 vol %, 55 vol %, 60 vol %, 65 vol %, 70 vol %, 90 vol % or between any porosities of above before being hardened. In some embodiments, composite materials (e.g., BRBC composite materials) cure in situ and have a porosity of more than 2 vol % or less than 15 vol % after being hardened. In some embodiments, a porosity of composite materials is 5 vol %, 10 vol %, 15 vol %, 20 vol %, 25 vol %, 30 vol %, 35 vol %, 40 vol %, 45 vol %, 50 vol %, 55 vol %, 60 vol %, 65 vol %, 70 vol %, 90 vol % or between any porosities of above after being hardened.

In some embodiments, in accordance with the present invention, composite materials (e.g., BRBC composite materials) that, when hardened, show a compressive strength within an approximate range of 4-10 MPa. In some embodiments, a compressive strength of such composite materials is about 2, 4, 6, 8, 10, 12, 14, 16, 18, 20, 22, 24, 26, 28, 30 MPa or between any compressive strengths of above. In some embodiments, a compressive strength is 17.5±6.2 MPa. In some embodiments, a compressive strength is 5.8±3.4 MPa.

In some embodiments, a compressive modulus of such composite materials is about 3800, 3600, 3400, 3200, 3000, 2500, 2000, 1500, 1000, 800, 600, 400, 200 MPa or between any compressive modulus of above. In some embodiments, a compressive modulus is 3230±936 MPa. In some embodiments, a compressive modulus is 1091±634 MPa.

In some embodiments, a shear modulus of such composite materials is about 400, 380, 360, 340, 320, 300, 280, 260, 240, 220, 200, 180, 160, 140, 120, 100 MPa or between any shear modulus of above. In some embodiments, a shear modulus is 289±140 MPa.

In some embodiments, an isotropic ultimate shear stress of such composite materials is about 1, 2, 4, 6, 8, 10, 12, 14, 16, 18, 20 MPa or between any isotropic ultimate shear stress of above. In some embodiments, an isotropic ultimate shear stress is 10.0±4.5 MPa.

In some embodiments, an ultimate torsional stress of such composite materials is about 1, 2, 3, 4, 5, 6, 7, 8, 9, 10 MPa or between any ultimate torsional stress of above. In some embodiments, an ultimate torsional stress is 6.1±2.7 MPa.

In some embodiments, it will be desirable to prepare composite materials whose porosity and/or relative proportion of particulate material (e.g., weight and/or volume percentages) is selected to achieve a particular remodel timing in relation to a particular set of mechanical properties.

For example, without wishing to be bound by any particular theory, it is proposed that composite materials with a volume percentage of particulate material within the approximate range of 50-60% and low porosity (e.g., below about 15%) are likely to remodel relatively quickly (e.g., degradation time less than 4, 8 or 12 weeks) and to exhibit mechanical properties suitable for trabecular bone in the femoral head. Alternatively or additionally, composite materials may have less than 50 vol % particles and low porosity (e.g., less than 15%). Such composite materials may remodel relatively slowly (e.g., scale with polymer degradation) and exhibit mechanical properties suitable for trabecular bone in femoral head.

Without wishing to be bound by any particular theory, it is also proposed that composite materials with less than 35 vol % particles and less than 40% porosity are likely to remodel relatively quickly (e.g., degradation time less than 4, 8 or 12 weeks) and to exhibit mechanical properties suitable for tibial plateau. Alternatively or additionally, composite materials may have less than 35 vol % particles and low porosity (e.g., less than 15%). Such composite materials may remodel relatively slowly (e.g., scale with polymer degradation) and exhibit mechanical properties suitable for tibial plateau.

### Preparation/Use of Composite Materials in Orthopedic Surgeries

According to the present invention, composite materials for use in orthopedic surgeries may be prepared in any of a variety of ways. In general, composite materials (e.g., BRBC composite materials) are prepared by combining particles, polymers and optionally any additional components. To form polyurethane-based composite materials (e.g., BRBC composite materials), for example, particles as discussed herein may be combined with a reactive liquid (i.e., a two-component composition of polyurethane) thereby forming a naturally injectable or moldable composite material or a material that can be made injectable or moldable. Alternatively, particles may be combined with polyisocyanate prepolymers or polyols first and then combined with other components.

In some embodiments, particles may be combined first with a hardener that includes polyols, water, catalysts and optionally a solvent, a diluent, a stabilizer, a porogen, a plasticizer, etc., and then combined with a polyisocyanate prepolymer. In some embodiments, a hardener (e.g., a polyol, water and a catalyst) may be mixed with a prepolymer, followed by addition of particles. In some embodiments, in order to enhance storage stability of two-component compositions, the two (liquid) component process may be modified to an alternative three (liquid)-component process wherein a catalyst and water may be dissolved in a solution separating from reactive polyols. For example, polyester polyols may be first mixed with a solution of a catalyst and water, followed by addition of bone particles, and finally addition of NCO-terminated prepolymers.

In some embodiments, additional components or components to be delivered may be combined with a reactive liquid prior to delivery (e.g., injection). In some embodiments, they may be combined with one of polymer precursors (i.e., prepolymers and polyols) prior to mixing the precursors in forming of a reactive liquid/paste.

Porous composites hardened from flowable composite materials (e.g., BRBC composite material) can be prepared by incorporating a small amount (e.g., <5 wt %) of water which reacts with prepolymers to form carbon dioxide, a biocompatible blowing agent. Resulting reactive liquid/paste may be injectable through a 12-ga syringe needle into molds or targeted site to set in situ. In some embodiments, gel time is great than 3 min, 4 min, 5 min, 6 min, 7 min, or 8 min. In some embodiments, cure time is less than 20 min, 18 min, 16 min, 14 min, 12 min, or 10 min.

In some embodiments, catalysts can be used to assist forming porous composites. In general, the more blowing catalyst used, higher porosity of composites may be achieved. In certain embodiments, surprisingly, surface demineralized bone particles may have a dramatic effect on the porosity. Without being bound to any theory, it is believed that the lower porosities achieved with surface demineralized bone particles in the absence of blowing catalysts result from adsorption of water to the hygroscopic demineralized layer on the surface of bones.

Polymers and particles may be combined by any method known to those skilled in the art to make composite materials. For example, a homogenous mixture of polymers and/or polymer precursors (e.g., prepolymers, polyols, etc.) and particles may be pressed together at ambient or elevated temperatures. At elevated temperatures, a process may also be accomplished without pressure. In some embodiments, polymers or precursors are not held at a temperature of greater than approximately 60° C. for a significant time during mixing to prevent thermal damage to any biological component (e.g., growth factors or cells) of a composite material. In some embodiments, temperature is not a concern because particles and polymer precursors used in the present invention have a low reaction exotherm.

Alternatively or in addition, particles may be mixed or folded into a polymer softened by heat or a solvent. Alternatively, a moldable polymer may be formed into a sheet that is then covered with a layer of particles. Particles may then be forced into the polymer sheet using pressure. In another embodiment, particles are individually coated with polymers or polymer precursors, for example, using a tumbler, spray coater, or a fluidized bed, before being mixed with a larger quantity of polymer. This facilitates even coating of particles and improves integration of particles and polymer component of a composite material.

After combination with particles, polymers may be further modified by further cross-linking or polymerization to form a composite material in which the polymer is covalently linked to the particles. In some embodiments, composition hardens in a solvent-free condition. In some embodiments, compositions are a polymer/solvent mixture that hardens when a solvent is removed (e.g., when a solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, a solvent is a biological fluid such as blood, plasma, serum, marrow, etc. In certain embodiments, a composite material is heated above the melting or glass transition temperature of one or more of its components and becomes set after implantation as it cools. In certain embodiments, a composite material is set by exposing a composite material to a heat source, or irradiating it with microwaves, IR rays, or UV light. Particles may also be mixed with a polymer that is sufficiently pliable to combine with the particles but that may require further treatment, for example, combination with a solvent or heating, to become a injectable or moldable composition. For example, a composition may be combined and injection molded, injected, extruded, laminated, sheet formed, foamed, or processed using other techniques known to those skilled in the art. In some embodiments, reaction injection molding methods, in which polymer precursors (e.g., polyisocyanate prepolymer, a polyol) are separately charged into a mold under precisely defined conditions, may be employed. For example, bone particles may be added to a precursor, or it may be separately charged into a mold and precursor materials added afterwards. Careful control of relative amounts of various components and reaction conditions may be desired to limit the amount of unreacted material in a composite. Post-cure processes known to those skilled in the art may also be employed. A partially polymerized polyurethane precursor may be more completely polymerized or cross-linked after combination with hydroxylated or aminated materials or included materials (e.g., a particulate, any components to deliver, etc.).

In some embodiments, as described herein, a composite material is prepared as a flowable or injectable composition and, then is set in situ.

In some embodiments, composite materials in a flowable or injectable state are characterized by viscosity below certain value, for example, 2000 Pa S. In some embodiments, composite materials in a flowable or injectable state are characterized by an ability to pass through needle of gauge 12. Without wishing to be bound by any particular theory, it is proposed that composite materials with less than 35 vol % particles are injectable through syringes.

In some embodiments, composite materials are considered to be “hardened” when viscosity is above certain value, for example, 2000 Pa S. In some embodiments, composite materials are hardened when certain strength (e.g., compressive modulus, compressive strength, shear modulus, isotropic ultimate shear stress, ultimate torsional stress, etc.) is achieved. For example, composite materials are hardened when their compressive strength is at least 1 or 2 MPa.

In some embodiments, a composite material transitions from a flowable/injectable state to a hardened state under physiological conditions including, for example, in the presence of an aqueous solution (e.g., saline, body fluid environment) and/or when exposed to a temperature of at least 37° C. or at least 40° C.

In some embodiments, a composite material transitions from a flowable/injectable state to a hardened state under physiological conditions over a time period of at less about 4, 5, 6, 7, 8, 9, 10, 12, 14, 16, 20 minutes or between any time of above.

In some embodiments, composite materials are prepared merely by combining relevant components with one another. In some embodiments, one or more additional processing steps is performed. In some such embodiments, the one or more additional processing steps may alter one or more properties of the composite material, or the state of the composite material.

For example, in some embodiments, cross-link density of a low molecular weight polymer may be increased by exposing it to electromagnetic radiation (e.g., UV light) or an alternative energy source. Alternatively or additionally, a photoactive cross-linking agent, chemical cross-linking agent, additional monomer, or combinations thereof may be mixed into composite materials. Exposure to UV light after a composition is injected into an implant site will increase one or both of molecular weight and cross-link density, stiffening polymers (i.e., polyurethanes) and thereby a composite material.

In some embodiments, polymer components of composite materials may be softened by a solvent, e.g., ethanol. In some embodiments, if a biocompatible solvent is used, a composite material is hardened in situ. In some embodiments, as a composite material sets (i.e., hardens), a solvent leaving such a hardened composite material is released into surrounding tissue without causing undesirable side effects such as irritation or an inflammatory response. In some embodiments, a composite material becomes moldable at an elevated temperature (e.g., 40° C.) into a pre-determined shape. In some embodiments, a composite material become hardened when applied in surgeries and allowed to cool to body temperature (approximately 37° C.).

The present invention provides methods of preparing composite materials by combining particle and polymer components. In some embodiments, methods are provided to combine bone particles and polyurethane precursors resulting in naturally flowable composite materials (e.g., BRBC composite materials). Alternatively or additionally, present invention provides methods to make a composite material (e.g., BRBC composite materials) include adding a solvent or pharmaceutically acceptable excipient to render a flowable or moldable composition (i.g., a flowable state). Such a composition may then be injected or placed into the site of implantation. As solvent or excipient diffuses out of the composite material, it may become set in place (i.g., a hardened state).

Polymer processing techniques may also be used to combine particles with a polyurethane or precursors (e.g., polyisocyanates and polyols). In some embodiments, polyurethanes and bone particles may be mixed in a solvent and cast with or without pressure. For example, a solvent may be dichloromethane. In some embodiments, a composition of particle and polymer utilized in the present invention is naturally injectable or moldable in a solvent-free condition.

In some embodiments, particles may be mixed with a polymer precursor according to standard composite processing techniques. For example, regularly shaped particles may simply be suspended in a precursor. A polymer precursor may be mechanically stirred to distribute the particles or bubbled with a gas, preferably one that is oxygen- and moisture-free. Once components of a composition are mixed, it may be desirable to store it in a container that imparts a static pressure to prevent separation of the particles and the polymer precursor, which may have different densities. In some embodiments, distribution and particle/polymer ratio may be optimized to produce at least one continuous path through a composite along particles.

Interaction of polymer components with particles may be enhanced by coating individual particles with a polymer precursor before combining them with bulk precursors. The coating enhances the association of a polymer component with particles. For example, individual particles may be spray coated with a monomer or prepolymer. Alternatively, the individual particles may be coated using a tumbler—particles and a solid polymer material are tumbled together to coat the particles. A fluidized bed coater may also be used to coat the particles. In addition, particles may simply be dipped into liquid or powdered polymer precursor. All of these techniques will be familiar to those skilled in the art.

In some embodiments, it may be desirable to infiltrate a polymer or polymer precursor into vascular and/or interstitial structure of bone particles or into bone-derived tissues. Vascular structure of bone includes such structures such as osteocyte lacunae, Haversian canals, Volksmann's canals, canaliculi and similar structures. Interstitial structure of bone particles includes spaces between trabeculae and similar features. Many of monomers and precursors (e.g., polyisocyanate prepolymers, polyols) suggested for use with the invention are sufficiently flowable to penetrate through the channels and pores of trabecular bone. Some may even penetrate into trabeculae or into mineralized fibrils of cortical bone. Thus, it may be necessary to incubate bone particles in polyurethane precursors for a period of time to accomplish infiltration. In certain embodiments, polyurethane itself is sufficiently flowable that it can penetrate channels and pores of bone. In certain embodiments, polyurethane may also be heated or combined with a solvent to make it more flowable for this purpose. Other ceramic materials and/or other bone-substitute materials employed as a particulate phase may also include porosity that can be infiltrated as described herein.

After implantation, hardened composite materials (e.g., BRBC composite materials) are allowed to remain at the site providing the strength desired while at the same time promoting healing of the bone and/or bone growth. Polyurethane of composite materials may be degraded or be resorbed as new bone is formed at the implantation site. Polymer may be resorbed over approximately 1 month to approximately 1 years. A composite material may start to be remodeled in as little as a week as it is infiltrated with cells or new bone in-growth. A remodeling process may continue for weeks, months, or years. In some embodiments, composite materials (e.g., BRBC composite materials) is resorbed and/or remodeled within about 4 weeks, 8 weeks, 12 weeks, 1 month, 2 months, 6 months, 8 months, 12 months, 1 year, 2 years, 5 years or between any time above.

One skilled in the art will recognize that standard experimental techniques may be used to test these properties for a range of compositions to optimize composite materials (e.g., BRBC composite materials) for a orthopedic surgery (e.g., joint replacement surgeries).

According to the present invention, composite materials (e.g., BRBC composite materials) of particular interest may be characterized by one or more properties such as mechanical properties and behaviors during handling. Composite materials (e.g., BRBC composite materials) can be prepared (e.g., mixed) and delivered (e.g., injected and/or applied) using standard techniques as well known in the art. In some embodiments, components of composite materials (e.g., BRBC composite materials) from a kit are mixed intraoperatively. Total operating time may not increase more than 20% as compared to PMMA. Composite materials (e.g., BRBC composite materials) may have a working time of 8-16 minutes, maintain handling properties (e.g., in a flowable state) for 2-5 minutes, and then harden quickly (e.g., in a hardened state). In some embodiments, a working time for composite materials (e.g., BRBC composite materials) is 6, 8, 9, 10, 11, 12, 14 or 16 minutes, or between any time above.

When fully combined and in a moldable or flowable state, composite materials (e.g., BRBC composite materials) may have a viscosity similar to that of PMMA and, in some embodiments, composite materials (e.g., BRBC composite materials) have a lower viscosity than that of Plexur M. In some embodiments, composite materials (e.g., BRBC composite materials) have a doughy consistency. An amount of time to set or harden composite materials (e.g., BRBC composite materials) may be similar to that of PMMA. Once hardened, in some embodiments, composite materials (e.g., BRBC composite materials) stay hard and not effected by additional application of heat, water, etc. In some embodiments, hardened composite materials (e.g., BRBC composite materials) provide initial fixation and maintains their strength to stabilize implants (e.g., a prosthesis) until remodeling occurs at interface of an implant and bone. In some embodiments, degradation of hardened composite materials (e.g., BRBC composite materials) is slow enough to not affect stiffness of an implant component (e.g., prosthesis) at an implant site. Alternatively or additionally, screws or metal backings are used for stabilization. Remodeling may take several months, less than a year, 1-2 years, more than two years, etc.

As discussed above, polymers or polymer precursors, and particles may be supplied separately, e.g., in a kit, and mixed immediately prior to implantation, injection or molding. A kit may contain a preset supply of bone particles having, e.g., certain sizes, shapes, and levels of demineralization. Surface of bone particles may have been optionally modified using one or more of techniques described herein. Alternatively, a kit may provide several different types of particles of varying sizes, shapes, and levels of demineralization and that may have been chemically modified in different ways. A surgeon or other health care professional may also combine components in a kit with autologous tissue derived during surgery or biopsy. For example, a surgeon may want to include autogenous tissue or cells, e.g., bone marrow or bone shavings generated while preparing a implant site, into a composite material (see more details in co-owned U.S. Pat. No. 7,291,345 and U.S. Ser. No. 11/625,119 published under No. 2007-0191963; both of which are incorporated herein by reference).

Composite materials (e.g., BRBC composite materials) utilized in accordance with the present invention may be optimized to use in a wide variety of surgical procedures. A method of preparing and using polyurethanes for various arthroplasties and revisions (e.g., TAK) utilized in the present invention may include the steps of providing a kit of a settable composite material (e.g., polyurethane-based BRBC composite materials), mixing parts of a composite material, and setting (hardening) a composite material in a implant site for initial fixation wherein such a composite material is sufficiently flowable to be applied initially and hardens later. In some embodiments, a flowable composite material to inject or apply may be pressed by hand or machine. In some embodiments, a moldable composite material may be pre-molded and implanted into a target site. Injectable or moldable compositions utilized in the present invention may be processed (e.g., mixed, pressed, molded, etc.) by hand or machine.

Composite materials (e.g., BRBC composite materials) may be used as injectable (e.g., initially flowable) materials with or without exhibiting high mechanical strength (i.e., load-bearing or non-load bearing, respectively). In some embodiments, composite materials (e.g., BRBC composite materials) are used as moldable materials. For example, compositions (e.g., prepolymer, monomers, reactive liquids/pastes, polymers, bone particles, additional components, etc.) in the present invention can be pre-molded into pre-determined shapes. Upon implantation, the pre-molded composite material may further cure in situ and provide mechanical strength (i.e., load-bearing).

In some embodiments, composite materials (e.g., BRBC composite materials) utilized in accordance with the present invention may be used for joint reconstruction, arthrodesis, arthroplasty, or cup arthroplasty of hips; for femoral or humeral head replacement; for femoral head surface replacement or total joint replacement; for repair of vertebral column, spinal fusion or internal vertebral fixation, etc. In some embodiments, composite materials (e.g., BRBC composite materials) are used as a bone void filler. In some embodiments, composite materials (e.g., BRBC composite materials) are used in TKA. In some embodiments, composite materials (e.g., BRBC composite materials) are highly effective in revision or complex total hip replacement, where acetabular bone is deficient.

In some embodiments, methods of utilizing composite materials (e.g., BRBC composite materials and other suitable cement materials) are similar to procedures used in primary TKA. First, a patient is placed on an operating table in a supine position. A sandbag can be placed under an ipsilateral hip to direct a knee perpendicular to the floor. After prepping and draping a leg, a thigh tourniquet is inflated. Femur, tibia, and patella are then exposed and shaped, and correct implant sizes are determined. A twelve-centimeter straight longitudinal incision can be made directly over a patella. Such an incision is then carried down through a fascia on the medial side of a knee, the patella is everted, and the knee joint is exposed and flexed. Using instrumentation, for example, provided by a orthopedic device company for prosthetic components (e.g., tibia components, femur components and patella components) or implants/devices (e.g., coated devices disclosed in U.S. Pat. No. 5,061,286) to be used, alignment is corrected and sequential cuts are made on tibia, femur, and patella. Cut surfaces can machined in accordance with specifics and geometrics of prosthetic components. Bony surfaces can be copiously irrigated in order to remove blood clot and soft tissue debris. This irrigation can facilitate the ability of a cement to penetrate and interdigitate with trabecular bone surfaces.

Composite materials (e.g., BRBC composite materials) utilized in accordance with the present invention are then prepared (e.g., mixed) and applied (e.g., by hand or machine). In some embodiments, after a composite material is initially prepared as a flowable, low viscosity mixture (e.g., similar to soft taffy), it can be applied to a tibial surface, and a tibial implant or component can be positioned in place engaged with the tibia until a composite material sets sufficiently. In some embodiments, composite materials (e.g., BRBC composite materials) can be finger packed into implant sites, while a delivery gun can also be used to inject a composite material in some embodiments. Flowability and low viscosity allow a composite material to integrate with trabeculae of bone. In some embodiments, when a composite material becomes more viscous (e.g., dough-like), it can be applied to a femur and a femur implant or component (where direct access to the bone may be limited), and a femur implant can be positioned engaged with the femur until a composite material sets (or hardens) sufficiently. In some embodiments, a composite material can be rolled into shape of a cigar and applied over prepared bone surfaces by hand. In some embodiments, a composite material is applied to a patella surface, and a patella implant or component can be positioned in place until a composite material sets (or hardens) sufficiently. In some embodiments, a patella implant is anchored by one or more tabs into the patella. Excess composite material, e.g., extruded from between bone and a implant during positioning, can be removed. In some embodiments, extra composite materials are left behind since it would not pose the same risk associate with PMMA. In some embodiments, a hardened composite material is chiseled off and irrigated. The knee joint is then copiously irrigated and the wound closed in layers using resorbable and non-resorbable sutures and/or staples of a surgeon's choice.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Using a Composite Material in a Total Knee Replacement

The patient in case #1 is a middle aged female with high BMI. The implant system is Stryker Triathlon and the composite material used herein is Simplex P Speedset (Stryker) “Faster Setting” (40 g powder/20 ml liquid).

Initial incisions were made followed by drilling axially into femur from the intercondylar region. A template for cutting/shaping the distal femur extends down the bone canal and is secured by screws. At this time, the angle of deformity (varus/valgus) may be corrected. More bone is removed from the lateral side of the femoral condyle. A first template on the proximal tibia was positioned. MLL was released to accommodate rebalancing and all osteophytes were removed. It was demonstrated that even with precise cuts, the fit for an implant is not tight and therefore, a surgeon may not rely on a press-fit (as with total hips) in TKA. The cement material was used to secure the implant to the bone, filling irregularities and gaps.

Implants were opened and prepared before mixing the cement material. At time zero, a cement material was mixed under vacuum to eliminate fumes and prevent bubbles. After approximately four minutes, the cement material became soft taffy, and was applied to the tibia first with a spatula (more flowable, less viscous consistency is desirable) so that the cement material was integrated with the trabeculae of the bone. Then excess cement material was removed after inserting a tibial implant component.

Three minutes later (approximately seven minutes from time zero), the cement material was applied to the femur (where a slightly more doughy feel is acceptable). The cement material was first rolled into a cigar shape and applied over to the prepared bone surfaces by hand. The cement material was also applied to the femur implant where access to bone surface is limited. An implant component is positioned, then removes excess was removed.

Approximately five minutes later (twelve minutes from time zero), the cement material was hot and hardening. Hardening rate may be effected by the room temperature, as well as by handling and heating. Heat is may not be involved. The maximum temperature may be lower.

The cement material was applied to a patella component, which is anchored by 3 tabs into the patella. The current standard for the patella component is to be made of polyethylene. Without wishing to be bound by any particular theory, it is proposed that bone would not grow into and integrate with the polyethylene. A metal backing may be necessary for use with a cement material.

The patella component is set into place after another two minutes (fourteen minutes from time zero). At this time, the cement material was hardened fully. All hardened cement material that has extruded out of the implant-bone space was carefully removed.

In case #2, the patient is a middle aged female of average BMI. BMI makes difference in visibility and ease of surgery. Similar cement materials and implants were used, and the same surgical procedure was followed as described above.

At time zero, a cement material was mixed under vacuum. For the first 1-2 minutes, the cement material was very liquid consistency was mixed in a chamber. The cement material was mixed with a spatula until it became more viscous. Three minutes later (approximately three minutes after time zero), the cement material was more viscous—the extent beyond which it may be difficult to inject. One minute later (approximately four minutes after time zero), the cement material exhibited good viscosity and was applied with spatula to tibial plateau. A tibial component was inserted and impacted. Excess cement material was removed.

Three minutes later (approximately seven minutes after time zero), the cement was placed onto a femoral component where access for application to the femur is limited. The doughy cement material was rolled into a cigar and placed on femur surfaces. Excess cement material was removed.

Four minutes later (approximately 11.25 minutes after time zero), the cement material was applied to the patella. A patella component was inserted. Excess cement material was removed.

The cement material was held to harden for another four minutes (approximately 15.5 minutes after time zero). Excess cement material was chisel away.

All references, such as patents, patent applications, and publications, referred to above are incorporated by reference in their entirety.

Still other embodiments are within the scope of the following claims.

1. In a joint arthroplasty comprising steps of:
accessing an implant area in a joint of a subject; and
applying a material within the implant area in an interface between a first bone and a first joint implant component;
the improvement comprising utilizing as the material a biocomposite remodeling bone cement (BRBC) composite material.

2. The method of claim 1, wherein the BRBC composite materials is prepared in a flowable state that is characterized by an ability to adopt both the flowable state and a hardened state.

3. The method of claim 2, wherein the BRBC composite materials is allowed to transition from the flowable state to the hardened state thereby fixing the first implant component to the first bone.

4. The method of claim 2, wherein the BRBC composite materials comprises a particulate component and a polymer component.

5. The method of claim 1, wherein the BRBC composite materials is characterized by an ability to remodel and induce bone ingrowth.

6. The method of claim 1, wherein the BRBC is characterized by an ability to adopt a plurality of flowable states having different viscosities.

7. The method of claim 1, further comprising a step of applying the composite between an second interface between a second bone and a second implant component.

8. The method of claim 7, wherein a first viscosity of the BRBC composite materials at the first interface is lower than a second viscosity of the BRBC composite materials at the second interface.

9. The method of claim 1, wherein the joint arthroplasty is a procedure selected from the group consisting of a total knee replacement surgical procedure, total shoulder replacement surgical procedure, complex total hip replacement surgical procedure, hip revision surgical procedure, knee revision surgical procedure, glenoid revision surgical procedure.

10. The method of claim 9, wherein the joint arthroplasty is a total knee arthroplasty.

11. The method of claim 7, wherein the first bone is a tibia, and the second bone is a femur.

12. The method of claim 11, further comprising a step of applying the BRBC composite materials to a patella.

13. The method of claim 1, wherein the BRBC composite materials is prepared by providing a first component and a second component.

14. The method of claim 13, wherein the first component and the second component are viscous fluids.

15. The method of claim 14, wherein the first component comprising a polyol.

16. The method of claim 14, wherein the second component comprising a polyisocyanate prepolymer.

17. The method of claim 13, wherein the BRBC composite materials is prepared by mixing with a third component.

18. The method of claim 17, wherein the third component is PMMA.

19. The method of claim 4, wherein the particulate component is selected from the group consisting of bone particles, bone substitutes or any combinations thereof.

20. The method of claim 1, wherein the BRBC composite materials is prepared at an elevated temperature.

21-30. (canceled)
